(19) United States
(12) Patent Application Publication
(10) Pub. No.: US 2010/0120078 A1
(43) Pub. Date: May 13, 2010

(54) Urine Stabilization System

(76) Inventor: Tony Baker, Sonora, CA (US)

Correspondence Address: King & Spalding LLP, 401 Congress Avenue, Suite 3200, Austin, TX, 78701, US

(21) Appl. No.: 12/569,542

(22) Filed: Sep. 29, 2009

Related U.S. Application Data
(63) Continuation-in-part of application No. 11/774,985, filed on Jul. 9, 2007.
Application No. 11/774,985 is a continuation-in-part of application No. 09/932,122, filed on Aug. 16, 2001, now Pat. No. 7,569,342.
Application No. 09/932,122 is a continuation of application No. 11/138,543, filed on May 25, 2005, now abandoned.
Application No. 11/138,543 is a continuation-in-part of application No. 12/048,961, filed on Mar. 14, 2008.
Application No. 12/048,961 is a continuation-in-part of application No. PCT/US07/63982, filed on Mar. 14, 2007.
(60) Provisional application No. 61/101,046, filed on Sep. 29, 2008.
(60) Provisional application No. 60/574,529, filed on May 25, 2004.
(60) Provisional application No. 60/894,795, filed on Mar. 14, 2007.
(60) Provisional application No. 60/971,881, filed on Sep. 12, 2007.
(60) Provisional application No. 60/983,468, filed on Oct. 29, 2007.

(51) Int. Cl. C12Q 1/02; C09K 15/16; G01N 31/00

(52) U.S. Cl. 435/29; 252/400.1; 436/15

(57) ABSTRACT

The present disclosure relates, according to some embodiments, to a method of stabilizing a molecule (e.g., a biomolecule) in a bodily fluid comprising: (1) providing a stabilizing solution comprising: (a) an amount of a divalent metal chelator selected from the group consisting of ethylenediaminetetraacetic acid (EDTA), (ethylenebis(oxyethylenenitrilo))tetraacetic acid (EGTA), and 1,2-bis(2-aminophenoxy)ethane-N,N,N′,N′-tetraacetic acid (BAPTA) and salts thereof in the range of from about 0.001 M to about 2 M; and (b) an amount of at least one chelator enhancing component selected from the group consisting of lithium chloride, guanidinium chloride, guanidinium thiocyanate, sodium salicylate, sodium perchlorate, and sodium thiocyanate in the range of from about 0.1 M to about 10 M; and (2) adding the stabilizing solution to the bodily fluid, thus stabilizing the molecule. A biomolecule may be selected from a nitrite, a carbohydrate, a ketone, a globin, a bilirubin, a lipid, and combinations thereof in some embodiments.

### CROSS-REFERENCE TO RELATED APPLICATIONS

This application claims priority to U.S. Provisional Application No. 61/101,046 filed Sep. 29, 2008, the entire contents of which are hereby incorporated in their entirety by this reference. This application is a continuation in part of U.S. application Ser. No. 11/774,985, filed Jul. 9, 2007, which is a continuation in part of U.S. application Ser. No. 09/932,122, filed Aug. 16, 2001 and which is a continuation of U.S. application Ser. No. 11/138,543, filed May 25, 2005, which claims priority to U.S. Provisional Application No. 60/574,529 filed May 24, 2004, the entire contents of all of which are hereby incorporated in their entirety by this reference. This application is a continuation in part of U.S. application Ser. No. 12/048,961, filed Mar. 14, 2008, which is a continuation in part of International PCT Application No. PCT/US07/63982 filed Mar. 14, 2007 and which claims the benefit of U.S. Provisional Patent Application Ser. No. 60/894,795, filed Mar. 14, 2007, U.S. Provisional Patent Application Ser. No. 60/970,881, filed Sep. 7, 2007, and U.S. Provisional Patent Application Ser. No. 60/983,468, filed Oct. 29, 2007, the entire contents of all of which are hereby incorporated in their entirety by this reference.

### FIELD OF THE INVENTION

The present disclosure relates, in some embodiments, to compositions and methods for the stabilization and/or stabilization of one or more macromolecules (e.g., proteins, nucleic acids, small molecules, and other analytes) in a bodily fluid (e.g., urine).

### BACKGROUND

Modern testing and treatment procedures have successfully reduced the prevalence and severity of many infectious diseases. For example, sexually-transmitted disease (STD) clinics regularly screen and treat patients for such diseases as gonorrhea and Syphilis. It is now well-known to identify infectious agents such as gonococci by analyzing a DNA sample. A genetic transformation test (GTT), such as Gonostat™, (Sierra Diagnostics, Inc., Sonora, Calif.), may be used to detect gonococcal DNA in specimens taken from the urethra of men, and the cervix and anus of women, according to Jaffe H W, Kraus S J, Edwards T A, Zubrzycki L. Diagnosis of gonorrhea using a genetic transformation test on mailed clinical specimens, J Inf Dis 1982; 146:275-279. A similar finding was also published in Whittington W L, Miller M, Lewis J, Parker J, Biddle J, Kraus S. Evaluation of the genetic transformation test, Abstr Ann Meeting Am Soc Microbiol 1983; p. 315.

The GTT is a test for biologically active or native DNA. For example, the Gonostat(3) GTT may be used to detect DNA such as gonococcal DNA in urine specimens. The Gonostat™ assay uses a test strain, Neisseria gonorrheae, ATCC 31953. This test strain is a mutant that is unable to grow into visible colonies on chocolate agar at 37° C. in 5% CO₂. Gonococcal DNA extracted from clinical material can restore colony growth ability to this test strain. The Gonostat™ assay is discussed in Zubrzycki L, Weinberger S S, Laboratory diagnosis of gonorrhea by a simple transformation test with a temperature-sensitive mutant of Neisseria gonorrhoeae. Sex Transm Dis 1980; 7:183-187.

It is not always possible to immediately test a patient for the presence of such an infectious agent. For example, clinical laboratories may not be readily found in many rural or underdeveloped areas. In such circumstances, it may be necessary to transport patient test specimens to a laboratory for analysis. Therefore, it may be desirable to stabilize such specimens for subsequent analysis with a GTT or other testing procedure.

Urine specimens may be practical and convenient for use in diagnoses of a medical condition (e.g., an infection, such as gonorrhea). A urine specimen may be collected by a patient, therefore avoiding the invasion of privacy and discomfort accompanying collection of other specimens, such as blood specimens, urethral cultures, or cervical cultures. Collection of a urine specimen by the patient also reduces the work load of the staff in the clinic or office.

DNA culture results of urine from males may be quite sensitive when the urine is cultured within two hours of collection. Such results may approach 92% to 94%, or even 100%, as described in Schachter J. Urine as a specimen for diagnosis of sexually transmitted diseases. Am J Med 1983; 75:93-97. However, the culture results of urine from females may not be very reliable, even when cultured within two hours. According to Schachter, only 47% to 73% of female urine cultures are positive relative to the culture results of cervical and anal specimens. Furthermore, culture results from any anatomic site may not be 100% sensitive. (See, for example, Johnson D W, Holmes K K, Kvale P A, Halverson C W, Hirsch W P. An evaluation of gonorrhea casefinding in the chronically infected male. Am J Epidemiol 1969; 90:438-448; Schmale J D, Martin J E, Domescik G. Observations on the culture diagnosis of gonorrhea in women. JAMA 1969; 210:213-314; Caldwell J G, Price E V, Pazin G J, Cornelius E C. Sensitivity and reproducibility of Thayer-Martin culture medium in diagnosing gonorrhea in women. Am J Gynecol 1971; 109:463-468; Kieth L, Moss W, Berger G S. Gonorrhea detection in a family planning clinic: A cost-benefit analysis of 2,000 triplicate cultures. Am J Obstet Gynecol 1975; 121:399-403; Luciano A A, Grubin L. Gonorrhea screening. JAMA 1980; 243:680-681; Goh B T, Varia K B, Ayliffe P F, Lim F K. Diagnosis of gonorrhea by gram-stained smears and cultures in men and women: Role of the urethral smear. Sex Trans Dis 1985; 12:135-139.

Currently, urine specimens must be tested quickly for the presence of some analytes (e.g., analytes subject to degradation, such as naked gonococcal DNA). Naked DNA may be intact double stranded DNA which is released from viable gonococci. Such naked DNA may be found in the urine of an infected patient. However, enzymes in urine rapidly destroy any DNA present in the specimen. The DNA may be either denatured, broken into single strands or totally destroyed by the enzymatic activity. This destruction of the DNA may effectively inactivate the naked gonococcal DNA for purposes of testing.

In a test such as the GTT, inactivation beyond the limits of detection may be determined by the inherent genetic needs for select gene sequences of the Gonostat mutant strain used in the Gonostat test. For example, the Gonostat transformation assay may be a very sensitive measurement tool for nucleic acid protection. In the GTT, the Gonostat organism must have approximately 1 picogram of native DNA to transform. This amount may be equal to the presence of approximately 30 gonorrhea bacteria in an inoculum. The average clinical infection has 10³-10⁵ such organisms.

The destruction of DNA by enzyme activity in a urine specimen increases with time. For example, naked gonococcal DNA in a urine specimen stored in excess of two hours is inactivated beyond the limits of detection of the GTT. As a result, testing of urine specimens for DNA may be very time-sensitive. For example, DNA-based tests such as the polymerase chain reaction (PCR), the ligase chain technology (LCₓ) test of Abbott Laboratories, Abbott Park, Ill., and the GTT all must be performed on a urine specimen within approximately two hours. FIG. 1 is a graph of DNA concentration in unstabilized urine according to the prior art, demonstrating DNA destruction over time. The gonococcal DNA concentrations of ten different types of urine specimens were tested using a GTT at hourly intervals, commencing one hour from time of inoculation. Approximately 200 transformants were counted at the one hour measurement. However, for all specimens, the number of transformants declined by more than 100% within one hour of this initial measurement. The number of transformants approached zero within the two hours of the initial measurement, FIG. 2 is a graph of eight day serial data on unstabilized urine according to the prior art, further illustrating DNA destruction in unstabilized samples. Approximately seven transformants were counted at the one day measurement. However, by the second day, testing indicated that the biologically active DNA in the unstabilized urine had been totally destroyed by enzyme activity.

Tests such as the GTT may also be used to detect DNA in such bodily fluids and excretions as blood, blood serum, amniotic fluid, spinal fluid, conjunctival fluid, salivary fluid, vaginal fluid, stool, seminal fluid, and sweat. FIG. 3 is a graph of DNA concentration in unstabilized serum according to the prior art, demonstrating DNA destruction over time. The gonococcal DNA concentrations of normal and abnormal serum of both male and female were tested at hourly intervals, commencing from the time of inoculation. Approximately 100 trans formants were counted at the one hour measurement. However, for all specimens, the number of transformants declined by more than 100% within three hours of this initial measurement. The number of transformants approached zero within the eight hours of the initial measurement.

Another test that may be used to identify DNA in a bodily fluid specimen is the PCR test. PCR testing uses discrete nucleic acid sequences and therefore may be effective even in the absence of intact DNA. FIG. 4 is a graph of PCR detection of MOMP Chlamydia in unstabilized urine according to the prior art, demonstrating DNA destruction over time. In PCR testing of an unstabilized urine specimen, four PCR absorbances were observed one hour after the addition of the MOMP Chlamydia. However, the number of PCR absorbances declined 100%, to two, when tested at two hours, and to zero by the third hour. This testing indicates that, even though PCR testing doesn't require intact DNA, the enzymatic activity of urine rapidly destroys even discrete nucleic acid sequences 45 within approximately three hours.

Unfortunately, practical and effective techniques for stabilizing DNA in certain bodily fluids have not been readily available. For example, one method used to deactivate urine enzymes is heating. In an experiment, urine was heated for five minutes in a boiling water bath (100° C.) and then cooled. Naked DNA and DNA released from gonococcal cells that were subsequently added to this urine were not deactivated. This suggests that the deoxyribonuclease component in urine may be a protein(s), since proteins may be denatured by such high temperatures.

However, heating may denature DNA that is already present in the urine specimen, including gonococcal DNA, as well as the DNA of Haemophilus influenzae and Bacillus subtilis. Heating may further denature or otherwise render undetectable proteins and other potential analytes. Thus, heating is not an appropriate method for stabilizing a patient urine specimen to test for the presence of such DNA. This is particularly true if the sample happens to be acidic, as heating DNA in an acidic medium may cause depurination, a reaction in which the purine bases are cleaved from the sugar-phosphate backbone. If depurination occurs, recognition reactions which depend for their specificity on the base sequence of the DNA become impossible.

In other known DNA assay systems, detergents or other chemicals may be added to assist in the detection of DNA. For example, in the DNA assay system described in Virtanen M, Syvanen A C, Oram J, Sodurlund H, Ranki M. Cytomegalovirus in urine: Detection of viral DNA by sandwich hybridization. J Clin Microbiol. 1984; 20:1083-1088, sarkosyl was used to detect cytomegalovirus (CMV) in urine by hybridization. In Boom R, Sol C J A, Salimans M M M, Jansen C L, Wertheim-van Dillen P M E, van der Noordaa J. Rapid and simple method for purification of nucleic acids. J Clin Microbiol 1990; 28:495-503, guanidinium chloride in urine was used to purify nucleic acids as assayed by gel electrophoresis. Although the reason for their use in these studies was not stated, the chemicals inactivated the deoxyribonuclease activity in urine that would have interfered with those assay systems.

### SUMMARY

Accordingly, a need has arisen for improved methods and systems for stabilizing and/or preserving (“stabilizing”) analytes (e.g., nucleic acids, proteins, small molecules, carbohydrates, lipids, and the like) in a bodily fluid such as urine, blood, blood serum, amniotic fluid, spinal fluid, conjunctival fluid, salivary fluid, vaginal fluid, stool, seminal fluid, and sweat, such that the efficacy of analyte assays, e.g., the PCR, LCₓ, and the GTT may be improved (e.g., optimized). If the primary sequence and/or three-dimensional structure of protein in a bodily fluid are stabilized, many specific assays, including immunoassays, ligand-receptor assays and enzyme assays, may be run. However, as emphasized above, proteins in such bodily fluids may be subject to rapid degradation. Such degradation may be carried by the ubiquitin system.

A need has also arisen for methods and systems for stabilizing small molecules in a bodily fluid, particularly urine. Many small molecules are participants in specific reactions, such as immunological reactions, antibody-antigen reactions, and/or reactions with receptors. Stabilizing biomolecules in a bodily fluid, therefore, may serve a number of purposes. For example, small molecules may be assayed for diagnosis of conditions associated with the presence or abnormal concentration of such an analyte (e.g., small molecule). The small molecules could also be assayed for forensic purposes, such as might be needed in the prosecution of rapes and other crimes of violence. In another example, urine biomolecules that attract animals (e.g., pheromones) may be stabilized (e.g., for hunting or fish bait) such that the activity of the pheromone may be stabilized.

The present disclosure relates, according to some embodiments, to methods and systems for stabilizing and/or preserving (“stabilizing”) analytes (e.g., nucleic acids, proteins, small molecules, carbohydrates, lipids, and the like) in a bodily fluid such as urine, blood, blood serum, amniotic fluid, spinal fluid, conjunctival fluid, salivary fluid, vaginal fluid, stool, seminal fluid, and sweat. One metric of stabilization, according to some embodiments, may include improved detection of the subject analyte, for example, in stabilized specimens versus unstabilized specimens. Methods and compositions, in some embodiments, may be readily usable and/or require a minimum of attention by the user. According to some embodiments, methods and compositions should also be capable of stabilizing proteins and small molecules for a significant period of time, even without refrigeration.

In some embodiments, methods, systems, and/or reagents of the disclosure may meet one or more of these need. A method of stabilizing a molecule selected from the group consisting of a protein and a small molecule in a bodily fluid, may comprise: (1) providing a stabilizing solution and/or (2) adding the stabilizing solution to the bodily fluid, thus stabilizing the molecule, wherein a stabilizing solution may comprise (a) an amount of a divalent metal chelator selected from the group consisting of ethylenediaminetetraacetic acid (EDTA), (ethylenebis(oxyethylenenitrilo))tetraacetic acid (EGTA), and 1,2-bis(2-aminophenoxy)ethane-N,N,N′,N′-tetraacetic acid (BAPTA) and salts thereof in the range of from about 0.001 M to about 2 M; and (b) an amount of at least one chelator enhancing component selected from the group consisting of lithium chloride, guanidinium chloride, guanidinium thiocyanate, sodium salicylate, sodium perchlorate, and sodium thiocyanate in the range of from about 0.1 M to about 10 M.

In some embodiments, a biomolecule may include a protein, for example, a protein selected from the group consisting of enzymes, antibodies, receptor proteins, regulatory proteins, membrane proteins, and structural proteins. Without limiting the disclosure to any particular mechanism of action, a protein may be protected from degradation by the ubiquitin system.

In some embodiments, a biomolecule may include a steroid, for example, a steroid selected from the group consisting of androsterone, testosterone, tetrahydrogestrinone, dehydrochlortestosterone, metandienone, methyltestosterone, androlone, oxandrolone, oxymetholone, stanozolol, and their analogues, precursors, and metabolites. A steroid (e.g., a stabilized steroid) may have (retain) pheromone activity according to some embodiments.

A bodily fluid may include, in some embodiments, urine, blood, serum, plasma, amniotic fluid, cerebrospinal fluid, seminal fluid, vaginal fluid, stool, conjunctival fluid, salivary fluid, and sweat. For example, a bodily fluid may be urine.

A biomolecule stabilized using a composition, system, and/or method of the disclosure may include a nitrite, a carbohydrate (e.g., a glycogen, a pentose, a hexose), a ketone (e.g., acetoacetate, acetone), a globin (e.g., myoglobin, hemoglobin), a bilirubin (e.g., urobilinogen, urobilin), a lipid, and combinations thereof. A carbohydrate may include, glucose, fructose, pyruvate, lactate, glycogen, and/or combinations thereof. In some embodiments, a biochemical property of a bodily fluid may be stabilized using a composition, system, and/or method of the disclosure including, for example, specific gravity, pH, pI, and/or the like. Cells that may be stabilized in a bodily fluid (e.g., urine), according to some embodiments, include blood cells (e.g., red blood cells, leukocytes).

A stabilizing composition may further include at least one enzyme inactivating component selected from the group consisting of manganese chloride, sarkosyl, and sodium dodecyl sulfate in the range of up to about 5% molar concentration.

According to some embodiments, a stabilizing composition may stabilize a molecule selected from a nitrite, a carbohydrate, a ketone, a globin, a bilirubin, a lipid, and combinations thereof. In some embodiments, a stabilizing composition may comprise (1) an amount of a divalent metal chelator selected from the group consisting of ethylenediaminetetraacetic acid (EDTA), (ethylenebis(oxyethylenenitrilo))tetraacetic acid (EGTA), and 1,2-bis(2-aminophenoxy)ethane-N,N,N′,N′-tetraacetic acid (BAPTA) and salts thereof in the range of from about 0.001 M to about 2 M; and (2) an amount of at least one chelator enhancing component selected from the group consisting of lithium chloride, guanidinium chloride, guanidinium thiocyanate, sodium salicylate, sodium perchlorate, and sodium thiocyanate in the range of from about 0.1 M to about 10 M.

The present disclosure, according to some embodiments, relates to a kit. For example, a kit may comprise (a) a stabilizing composition (e.g., as described above), (b) a vessel for collecting a biological fluid in which a protein or small molecule is to be stabilized, and/or (c) instructions for use.

In some embodiments, a composition may comprise animal urine and a stabilizing composition wherein the animal urine contains a pheromone in sufficient quantity to act as an attractant to an animal of the same species as the animal from which the animal urine comes. A method of stabilizing pheromone activity of an animal urine, in some embodiments, may comprise (1) providing a fresh animal urine containing pheromone activity; and (2) adding the fresh animal urine to a stabilizing composition of the present disclosure as described above to stabilize the pheromone activity at a level such that the urine containing a stabilizing composition acts as an attractant to an animal of the same species as the animal from which the animal urine comes.

A stabilized fluid may comprise, in some embodiments, (1) a stabilizing composition for stabilizing a molecule selected from the group consisting of a a nitrite, a carbohydrate, a ketone, a globin, a bilirubin, a lipid, and combinations thereof and (2) a bodily fluid from a human or non-human subject (e.g., a primate), wherein a stabilizing composition may comprise (a) an amount of a divalent metal chelator selected from the group consisting of ethylenediaminetetraacetic acid (EDTA), (ethylenebis(oxyethylenenitrilo))tetraacetic acid (EGTA), and 1,2-bis(2-aminophenoxy)ethane-N,N,N′,N′-tetra acetic acid (BAPTA) and salts thereof in the range of from about 0.001 M to about 2 M; and (b) an amount of at least one chelator enhancing component selected from the group consisting of lithium chloride, guanidinium chloride, guanidinium thiocyanate, sodium salicylate, sodium perchlorate, and sodium thiocyanate in the range of from about 0.1 M to about 10 M.

### BRIEF DESCRIPTION OF THE DRAWINGS

Some embodiments of the disclosure may be understood by referring, in part, to the present disclosure and the accompanying drawings, wherein:

FIG. 1 is a graph of DNA concentration in unstabilized urine according to the prior art.

FIG. 2 is a graph of eight day serial data on unstabilized urine according to the prior art.

FIG. 3 is a graph of DNA concentration in unstabilized serum according to the prior art.

FIG. 4 is a graph of PCR detection of MOMP Chlamydia in unstabilized urine according to the prior art.

FIG. 5 is a bar graph of DNA concentration in stabilized urine according to some embodiments of the disclosure.

FIG. 6 is a graph of eight day serial data on stabilized urine according to some embodiments of the disclosure.

FIG. 7 is a graph comparing PCR results in unstabilized and stabilized normal urine according to some embodiments of the disclosure.

FIG. 8 is a graph of eight day serial data on stabilized serum according to some embodiments of the disclosure.

FIG. 9 is a graph of DNA concentration in stabilized serum according to some embodiments of the disclosure.

FIG. 10 is a flow chart of a method for stabilizing DNA according to a specific example embodiment of the disclosure.

FIG. 11 is a diagram of a system for stabilizing DNA according to a specific example embodiment of the disclosure.

FIG. 12 graphically illustrates a comparison of signal response in PCR assays wherein the DNA has been treated with a stabilizing composition according to some embodiments of the disclosure, and one which has not.

FIG. 13 illustrates the efficacy of reagents of the present disclosure to enhance signal response of a branched DNA assay of blood plasma samples subjected to various storage conditions.

FIG. 14 illustrates the efficacy of reagents of the present disclosure to enhance signal response of a branched DNA assay of blood serum and plasma samples.

FIG. 15 is a graph showing the interference of methemoglobin on PCR absorbance in a PCR amplification assay on hepatitis B sequences MD03/06 in unprotected serum;

FIG. 16 is a graph showing the improvement in attenuating the interference of methemoglobin on PCR absorbance in a PCR amplification assay on hepatitis B sequences MD03/06 in serum which has been treated with a stabilizing composition according to some embodiments of the disclosure.

FIG. 17A illustrates the synergistic effect provided by the components of the inventive reagents in protecting hepatitis B sequences in serum stored at room temperature and subsequently subjected to MD03/06 PCR detection.

FIG. 17B illustrates the synergistic effect provided by the components of the inventive reagents in protecting hepatitis B sequences in serum stored at room temperature and subsequently subjected to MD03/06 PCR detection.

FIG. 17C illustrates the synergistic effect provided by the components of the inventive reagents in protecting hepatitis B sequences in serum stored at room temperature and subsequently subjected to MD03/06 PCR detection.

FIG. 17D illustrates the synergistic effect provided by the components of the inventive reagents in protecting hepatitis B sequences in serum stored at room temperature and subsequently subjected to MD03/06 PCR detection.

FIG. 17E illustrates the synergistic effect provided by the components of the inventive reagents in protecting hepatitis B sequences in serum stored at room temperature and subsequently subjected to MD03/06 PCR detection.

FIG. 17F illustrates the synergistic effect provided by the components of the inventive reagents in protecting hepatitis B sequences in serum stored at room temperature and subsequently subjected to MD03/06 PCR detection.

FIGS. 18A-18F are graphs showing the absence of a stabilizing effect that some individual components have on gonococcal DNA in urine stored at room temperature and subsequently subjected to PCR detection.

FIG. 19A is a graph showing comparative stabilization of androsterone in androsterone-spiked human urine over 12 months using a specific example embodiment of a composition comprising guanidinium HCl/EDTA versus potassium acid phosphate.

FIG. 19B is a graph showing comparative stabilization of androsterone in androsterone-spiked human urine over 12 months using a specific example embodiment of a composition comprising guanidinium HCl/EDTA versus boric acid.

FIG. 19C is a graph showing comparative stabilization of androsterone in androsterone-spiked human urine over 12 months using a specific example embodiment of a composition comprising guanidinium HCl/EDTA versus sodium bicarbonate.

FIG. 19D is a graph showing comparative stabilization of androsterone in androsterone-spiked human urine over 12 months using a specific example embodiment of a composition comprising guanidinium HCl/EDTA versus benzoic acid.

FIG. 19E is a graph showing comparative stabilization of androsterone in androsterone-spiked human urine over 12 months using a specific example embodiment of a composition comprising guanidinium HCl/EDTA versus sodium benzoate.

FIG. 20 is a graph showing the prevention of degradation of protein AF176555 (calpain) in urine by the ubiquitin-28S proteasome pathway using single agents and combination agents; with chaotropic agents used at 2 M and chelators at 0.1 M. The single agents were sodium thiocyanate, guanidinium thiocyanate, guanidinium HCl, sodium perchlorate, and EDTA. The combination agents were sodium thiocyanate+EDTA, guanidinium thiocyanate+EDTA, guanidinium HCl+EDTA, sodium perchlorate+EDTA, and lithium chloride+EDTA.

FIG. 21 is a graph showing the survival of ubiquitin activating enzymes Ubc2 (E-2) and Ubc3 (E-2) in urine with and without 2M sodium thiocyanate and 0.1 M EDTA.

FIG. 22 is a graph showing the survival of protein AF068706 (G2AD) from degradation by the ubiquitin system in urine spiked with ubiquitin, activating enzymes E-1, E-2, E-3, ATP, and 28S proteasome by 2 M sodium thiocyanate+0.1 M EDTA compared with frozen controls and unprotected protein.

FIG. 23 is a graph showing the survival of Protein NM_{—}015416 (cervical cancer proto-oncogene protein p40) from degradation by the ubiquitin system in urine spiked with ubiquitin, activating enzymes E-1, E-2, E-3, ATP, and 28S proteasome by 2 M sodium thiocyanate+0.1 M EDTA compared with frozen controls and unprotected protein.

FIG. 24 is a graph showing the survival of ATP in urine with and without exposure to 2 M sodium thiocyanate+0.1 M EDTA.

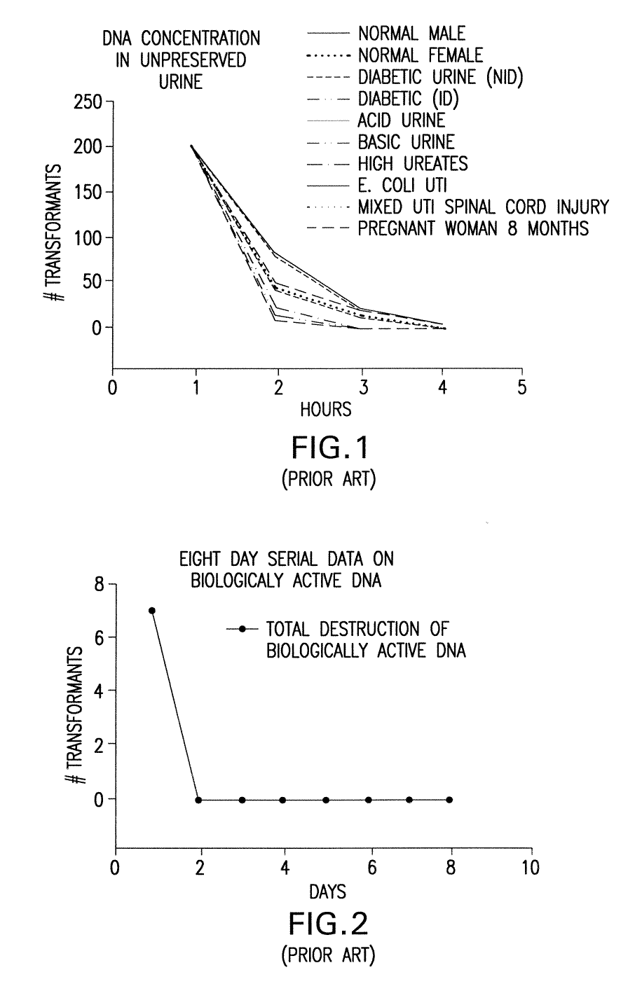

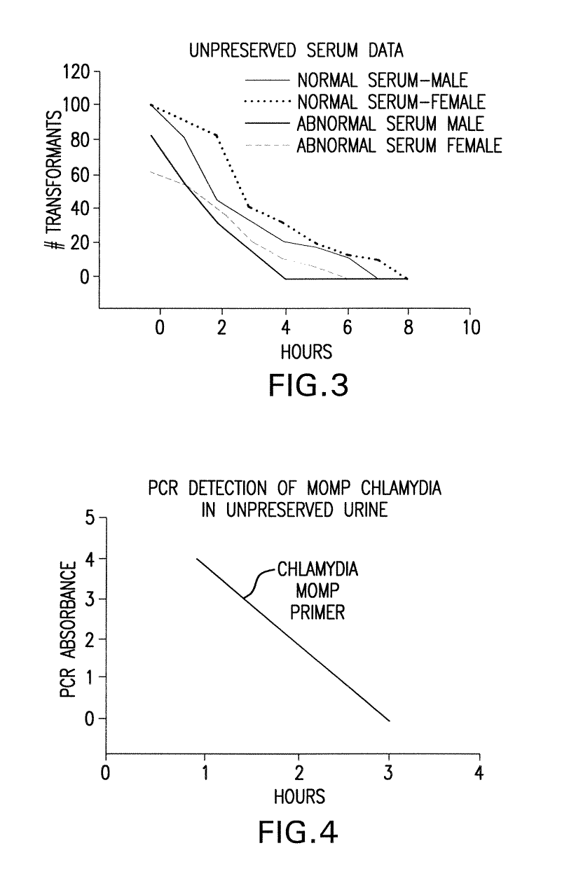

### DETAILED DESCRIPTION

The present disclosure relates, in some embodiments, to improved methods, systems and reagents for stabilizing nucleic acids, e.g., DNA and RNA; proteins; and small molecules in bodily fluids. Small molecules may include, without limitation, compounds that may act as pheromones, such as steroids, either free or complexed with proteins. Methods, systems and/or reagents of the disclosure may enable, in some embodiments, one or more molecular assays of nucleic acids, proteins, or small molecules in a bodily fluid and/or excretions. Examples of a bodily fluid may include, without limitation, blood, blood serum, amniotic fluid, spinal fluid, conjunctival fluid, salivary fluid, vaginal fluid, stool, seminal fluid, and sweat. In some embodiments, these molecular assays may be carried out with greater sensitivity. Methods, systems and reagents according to some embodiments have been found to surprisingly increase the signal obtained with such nucleic acid testing methods as the polymerase chain reaction (PCR), LCₓ, and genetic transformation testing (GTT). In particular, some embodiments the disclosure has also been found to surprisingly modulate the effect of hemoglobin, e.g., methemoglobin, interference on nucleic acid assays such as PCR on serum samples. Additionally, hybridization in such nucleic acid testing methods is unexpectedly improved. The specification of U.S. Pat. No. 6,458,546 to Baker is incorporated herein by this reference.

In some embodiments, the disclosure relates to methods of stabilizing a nucleic acid in a fluid such as a bodily fluid, including providing a nucleic acid stabilizing solution and adding the nucleic acid stabilizing composition to a fluid, e.g., a bodily fluid, wherein a stabilizing composition may comprise an amount of a divalent metal chelator selected from ethylenediaminetetraacetic acid (EDTA), [ethylenebis(oxyethylenenitrilo)] tetraacetic acid (EGTA) and 1,2-bis(2-aminophenoxy)ethane-N,N,N′,N′-tetraacetic acid (BAPTA), and salts thereof; and an amount of at least one chelator enhancing component selected from lithium chloride, guanidine, sodium salicylate, sodium perchlorate, and sodium thiocyanate. According to some embodiments, the amount of a divalent metal chelator may be in the range of from about 0.001M to 0.1M and/or The amount of a chelator enhancing component may be in the range of from about 0.1M to 2M. The amount of chelator enhancing component may be at least 1M in a stabilizing solution, and a divalent metal chelator may be present in an amount of at least about 0.01M.

A method for stabilizing a protein or a small molecule (e.g., a compound acting as a pheromone) may comprise, in some embodiments, (a) providing a stabilizing solution comprising an amount of a divalent metal chelator selected from ethylenediaminetetraacetictic acid (EDTA), [ethylenebis(oxyethylenenitrilo)]tetraacetic acid (EGTA) and 1,2-bis(2-aminophenoxy)ethane-N,N,N′,N′-tetraacetic acid (BAPTA), and salts thereof; and an amount of at least one chelator enhancing component selected from lithium chloride, guanidinium chloride, guanidinium thiocyanate, sodium salicylate, sodium perchlorate, and sodium thiocyanate; and/or (b) adding the stabilizing solution to a fluid, e.g., a bodily fluid. In some embodiments, the amount of a divalent metal chelator may be in the range of from about 0.001 M to 2 M and/or The amount of a chelator enhancing component may be in the range of from about 0.1M to 10 M. The amount of chelator enhancing component may be at least 1 M in a stabilizing solution, and a divalent metal chelator may be present in an amount of at least about 0.01 M, particularly when the stabilization of proteins or small molecules is desired. In some embodiments, a bodily fluid may be urine, but may be another bodily fluid as described below. A bodily fluid may be a bodily fluid from a human subject, or a bodily fluid from a non-human animal, such as a socially or economically important animal such as a cow, a goat, a sheep, a pig, a dog, a horse, or a cat, or an animal that is hunted or tracked, such as a deer, a fox, a bear, a boar, an elk, a moose, or a raccoon. Human bodily fluid may be used with diagnostic or forensic applications as discussed below.

In some embodiments, when stabilizing a protein or a small molecule, such as a compound acting as a pheromone, the amount of a divalent metal chelator may be increased so that it is in the range of from about 0.001 M to about 2 M. Similarly, the amount of a chelator enhancing component may be increased so that it is in the range of from about 0.1 M to about 10 M. These concentrations may be adjusted as desired and/or required. For example, when stabilizing a nucleic acid, it may be desirable and/or required to use concentrations of divalent metal chelator and chelator enhancing component low enough so that there is substantially no interference with a nucleic-acid-hybridization-dependent assay such as PCR. On the other hand, when stabilizing a protein or a small molecule, it may not be necessary to use such low concentrations of divalent metal chelator and chelator enhancing component. As indicated above, the amount of chelator enhancing component may be at least 1 M in a stabilizing solution, and a divalent metal chelator may be present in an amount of at least about 0.01 M, particularly when the stabilization of proteins or small molecules is desired.

According to some embodiments, a stabilizing composition for stabilizing a molecule selected from the group consisting of a protein and a small molecule comprising (1) an amount of a divalent metal chelator selected from the group consisting of ethylenediaminetetraacetic acid (EDTA), (ethylenebis(oxyethylenenitrilo))tetraacetic acid (EGTA), and 1,2-bis(2-aminophenoxy)ethane-N,N,N′,N′-tetraacetic acid (BAPTA) and salts thereof in the range of from about 0.001 M to about 2 M, and (2) an amount of at least one chelator enhancing component selected from the group consisting of lithium chloride, guanidinium chloride, guanidinium thiocyanate, sodium salicylate, sodium perchlorate, and sodium thiocyanate in the range of from about 0.1 M to about 10 M.

As indicated above, a stabilizing composition may further comprise at least one enzyme inactivating component selected from the group consisting of manganese chloride, sarkosyl, and sodium dodecyl sulfate in the range of up to about 5% molar concentration.

As also indicated above, the amount of chelator enhancing component may be at least 1 M in a stabilizing solution, and a divalent metal chelator may be present in an amount of at least about 0.01 M in a stabilizing solution, particularly when the stabilization of proteins or small molecules is desired.

A method for stabilizing a protein may include, in some embodiments, removing (partially or completely) divalent metal chelator and/or chelator enhancing component, for example, where a stabilized protein is to be subjected to an assay (e.g., an immunoassay) that is impacted (e.g., adversely) by the presence of a divalent metal chelator and/or a chelator enhancing component. A divalent metal chelator and/or a chelator enhancing component may be removed, for example, by methods known in the art, such as equilibrium dialysis (e.g., against a buffer containing lower concentrations of divalent metal chelator and chelator enhancing component or lacking these components) and/or lyophilization followed by reconstitution in a desired buffer.

In some embodiments, the disclosure relates to stabilizing solutions comprising an amount of a divalent metal chelator selected from EDTA, EGTA and BAPTA, and salts thereof; and an amount of at least one chelator enhancing component selected from lithium chloride, guanidinium chloride, guanidinium thiocyanate, sodium salicylate, sodium perchlorate, and sodium thiocyanate. Stabilizing solutions according to the disclosure may be formulated to stabilize nucleic acids, proteins, or small molecules such as steroids. When a stabilizing solution is formulated to stabilize nucleic acids, the amount of a divalent metal chelator may be in the range of from about 0.001 M to 0.1 M and/or The amount of a chelator enhancing component may be in the range of from about 0.1 M to 2 M according to some embodiments. When a stabilizing solution is formulated to stabilize nucleic acids, the amount of chelator enhancing component may be at least 1 M in a stabilizing solution, and a divalent metal chelator may be present in an amount of at least about 0.01 M.

When a stabilizing solution is formulated to stabilize proteins or small molecules, the amount of a divalent metal chelator may be in the range from about 0.001 M to about 2 M and/or The amount of a chelator enhancing component may be in the range of from about 0.1 M to about 10 M.

Methods, systems, and/or reagents (e.g., stabilizing compositions) of the disclosure may further include an amount of at least one enzyme inactivating component such as manganese chloride, sarkosyl, or sodium dodecyl sulfate, generally in the range of up to about 5% molar concentration.

In some embodiments the disclosure relates to a method of improving the signal response of a molecular assay of a test sample, including providing a stabilizing solution comprising an amount of a divalent metal chelator selected from EDTA, EGTA and BAPTA, and salts thereof; and an amount of at least one chelator enhancing component selected from lithium chloride, guanidine, sodium salicylate, sodium perchlorate, and sodium thiocyanate; adding a stabilizing composition to a test sample to provide a stabilized test sample; extracting molecular analytes of interest, e.g., DNA, RNA, proteins, or small molecules such as steroids from the stabilized test sample, and conducting a molecular assay on the extracted molecular analytes of interest. The amount of a divalent metal chelator may be generally as described above, e.g. in the range of from about 0.001 M to 0.1 M when the molecular analyte of interest is DNA or RNA, or in the range of from about 0.001 M to about 2 M when the molecular analyte of interest is a protein or a small molecule. Similarly, the amount of a chelator enhancing component may be generally as described above, e.g. in the range of from about 0.1 M to 2 M when the molecular analyte of interest is DNA or RNA, or in the range of from about 0.1 M to about 10 M when the molecular analyte of interest is a protein or a small molecule. A chelator enhancing component may comprise, in some embodiments, one or more of sodium perchlorate, sodium thiocyanate, sodium perchlorate, guanidine, and lithium chloride. The amount of chelator enhancing component may be at least 1 M in a stabilizing solution, and a divalent metal chelator may be present in an amount of at least about 0.01 M. Without limiting any particular embodiment(s) to any specific mechanism(s) of action, signal response of nucleic acid assays may be enhanced, in part, due to enhanced hybridization between probe and target molecules in the presence of a stabilization reagent of the disclosure.

According to some embodiments, methods, systems, and/or reagents of the disclosure for stabilizing a nucleic acid may eliminate enzymatic destruction of the nucleic acid of interest in a bodily fluid. A stabilizing composition may optionally be provided in solid or gaseous forms. While methods, systems, and/or reagents of the disclosure may be useful in stabilizing all types of nucleic acids, e.g., RNA and DNA, including human DNA, and bacterial, fungal, and viral DNA, some embodiments of the disclosure may be especially advantageous for use in stabilizing prokaryotic DNA, e.g., gonococcal DNA, DNA of Haemophilus influenzae and Bacillus subtilis.

Nucleic acids in a bodily fluid may be stabilized for testing for a significantly longer period of time than that permitted by other methods and compositions. While the maximum time between collecting, mailing, and testing patient specimens may be expected to be approximately six days, some embodiments of the disclosure may be effective beyond that period of time.

Stabilizing compositions of the disclosure may be used advantageously to stabilize prokaryotic, e.g., gonococcal DNA, according to some embodiments. In some embodiments, stabilizing compositions of the disclosure may be applied to the stabilization of other types of DNA, including human, bacterial, fungal, and viral DNA, as well as to RNA. Without limiting any particular embodiment(s) to any specific mechanism(s) of action, reagents of the disclosure may function by inactivating one or members of two classes of enzymes that may be present in bodily fluids (e.g., blood or urine) and may be reduce nucleic acid integrity, namely metal-dependent and metal independent enzymes. For example, a divalent metal chelator may reduce and/or remove one or more divalent metals (e.g., magnesium and calcium cation (Mg⁺², Ca⁺²)), which may effectively inactivate metal dependent enzymes such as deoxyribonucleases. A component of deoxyribonucleases has been found to inactivate gonococcal DNA in unstabilized urine. Non-limiting examples of a divalent metal chelator may include ethylenediaminetetraacetic acid (EDTA), [ethylenebis(oxyethylenenitrilo)]tetraacetic acid (EGTA), or 1,2-bis(2-aminophenoxy)ethane-N,N,N′,N′-tetraacetic acid (BAPTA), or salts thereof. The amount of a divalent metal chelator may be in the range of from about 0.001M to 0.1M when stabilizing solutions according to the present disclosure are used to stabilize nucleic acids. In some embodiments, the amount of a divalent metal chelator in a stabilizing solution is at least 0.01M.

In some embodiments, a chelator enhancing component may assist a divalent metal chelator in protecting the nucleic acids in a fluid. Without limiting any particular embodiment(s) to any specific mechanism(s) of action, a chelator enhancing component may inactivate metal independent enzymes found in bodily fluids such as DNA ligases (e.g., D4 DNA ligase), DNA polymerases (e.g., T7 DNA polymerase), exonucleases (e.g., exonuclease 2,2-exonuclease), kinases (e.g., T4 polynucleotide kinase), phosphatases (e.g., BAP and CIP phosphatase), nucleases (e.g., BL31 nuclease, and XO nuclease), and RNA-modifying enzymes (e.g., E. coli RNA polymerase, SP6, T7, T3 RNA polymerase, and T4 RNA ligase). Lithium chloride, guanidinium chloride, guanidinium thiocyanate, sodium salicylate, sodium perchlorate, and sodium thiocyanate have been found to be particularly effective under conditions examined The amount of a chelator enhancing component may be in the range of from about 0.1 M to 2 M when stabilizing solutions according to the present disclosure are used to stabilize nucleic acids. In some embodiments the amount of chelator enhancing component in a stabilizing solution is at least 1 M.

According to some embodiments, methods, systems, and/or reagents of the disclosure may surprisingly increase the signal obtained with such nucleic acid testing methods as the polymerase chain reaction (PCR), LCₓ, and genetic transformation testing (GTT). For example, some embodiments have been found to surprisingly and unexpectedly enhance hybridization in nucleic acid testing methods such as the PCR. FIG. 12 illustrates improvement in hybridization obtained by use of a composition disclosed herein on the hybridization of penicillinase-producing Neisseria gonorrheae (PPNG) DNA and PPNG-C probe.

According to some embodiments, the disclosure relates to methods of improving hybridization of nucleic acids, including contacting a test nucleic acid with a nucleic acid stabilizing solution comprising an amount of a divalent metal chelator selected from ethylenediaminetetraacetic acid (EDTA), ethylenebis(oxyethylenenitrilo)]tetraacetic acid (EGTA) and 1,2-bis(2-aminophenoxy)ethane-N,N,N′,N′-tetraacetic acid (BAPTA), or salts thereof in the range of from about 0.001 M to 0.1 M; and an amount of at least one chelator enhancing component selected from lithium chloride, guanidinium chloride, guanidinium thiocyanate, sodium salicylate, sodium perchlorate, and sodium thiocyanate in the range of from about 0.1 M to 2 M, such that a test solution is formed; and contacting the test solution with a target nucleic acid under conditions favorable for hybridization, such that hybridization occurs.

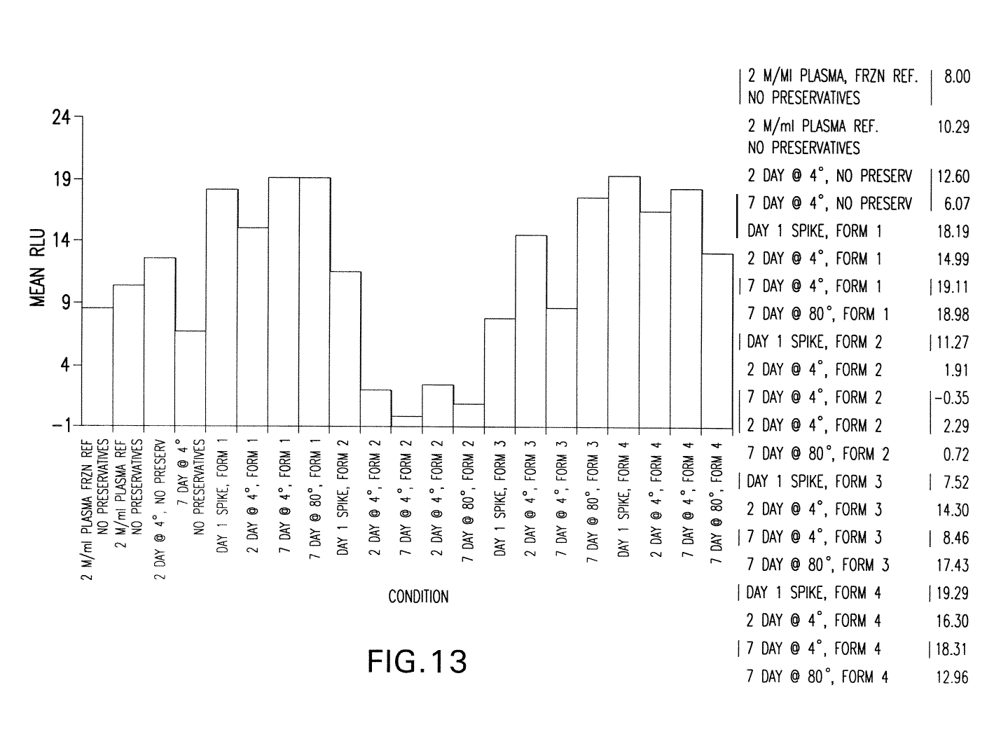

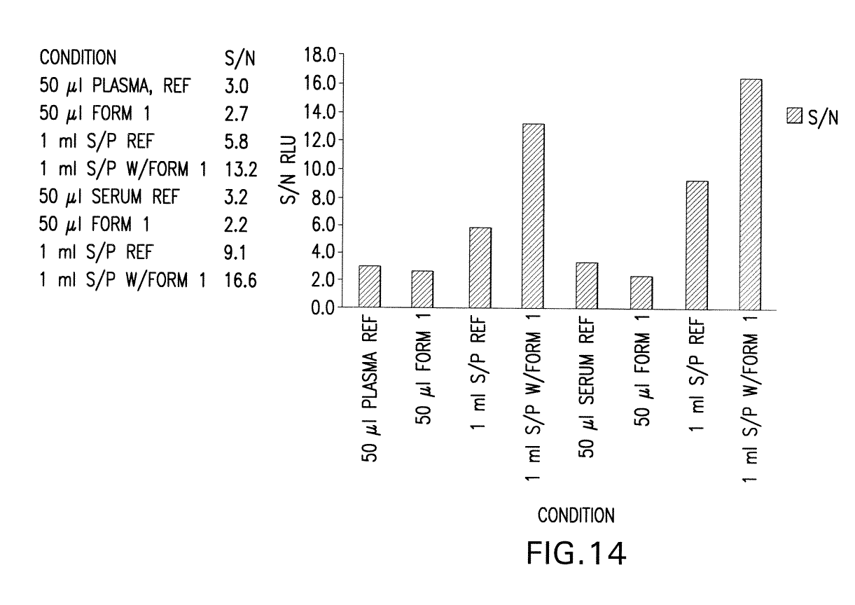

FIG. 13 and FIG. 14 further illustrate the efficacy of methods, systems, and/or reagents of the disclosure in improving the results obtained with nucleic acid testing methods, in this case, a branched DNA (bDNA) assay (Chiron). In the tests run in FIG. 13, the bDNA assay was used to assess the protective effect of the DNA/RNA protect reagents. DNA sequences from the hepatitis C virus were spiked into serum and plasma. The protected serum and plasma were mixed with 9 ml of serum or plasma and 1 ml of stabilizing composition. The following formulations were used: 1) 1 M guanidine HCl/0.01 M EDTA, 2) 1 M sodium perchlorate/0.01 M BAPTA, 3) 1 M sodium thiocyanate/0.01 M EGTA, and 4) 1 M lithium chloride/0.01 M EGTA. The formulations were stored for seven days at 4° C. The bDNA assay relies on hybridization; it can clearly be seen from the absorbance results that the target sequences were not only protected against degradation, but the more than doubling of the absorbance results indicates an enhancement of hybridization/annealing of the target sequences.

FIG. 14 illustrates a serum versus plasma study in which 50 μl samples of fresh human plasma, and 1 ml samples of fresh human serum were protected with 1 M guanidine HCl/0.01 M EDTA and the bDNA assay was run on these samples after the samples were stored at 20° C. for 48 hours. Results were compared to unprotected samples. It can clearly be seen from the absorbance results that the target sequences were not only protected against degradation, but the more than doubling of the absorbance results indicates an enhancement of hybridization/annealing of the target sequences.

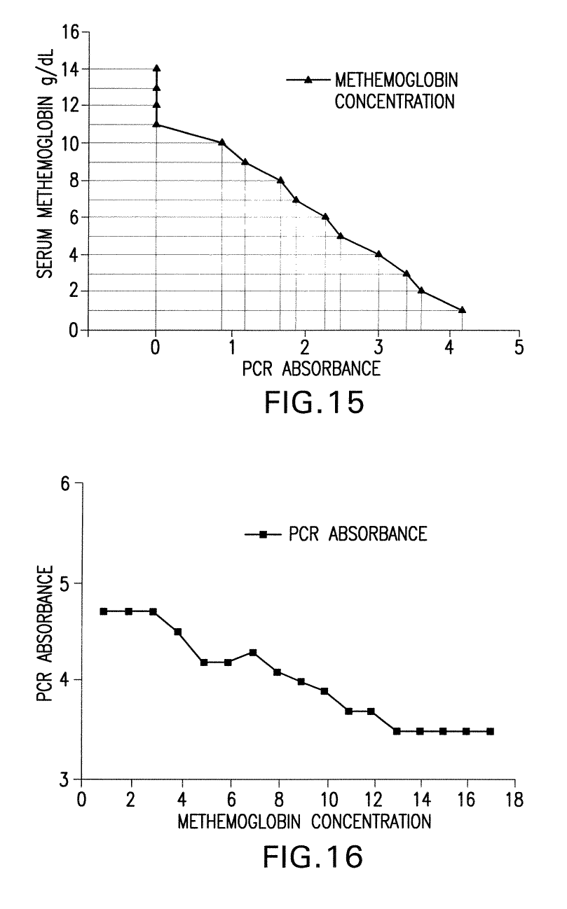

Stabilizing composition reagents of the disclosure have also surprisingly been found to remove the interference with heme compounds, e.g., methemoglobin, on PCR assays run on blood serum. FIG. 15 and FIG. 16 illustrate the improvement obtained by use of some examples of stabilizing compositions disclosed herein. Increasing amounts of methemoglobin were spiked into unprotected fresh human serum, to a concentration of 10 dl/ml. Serial PCR assays were run over a four hour period.

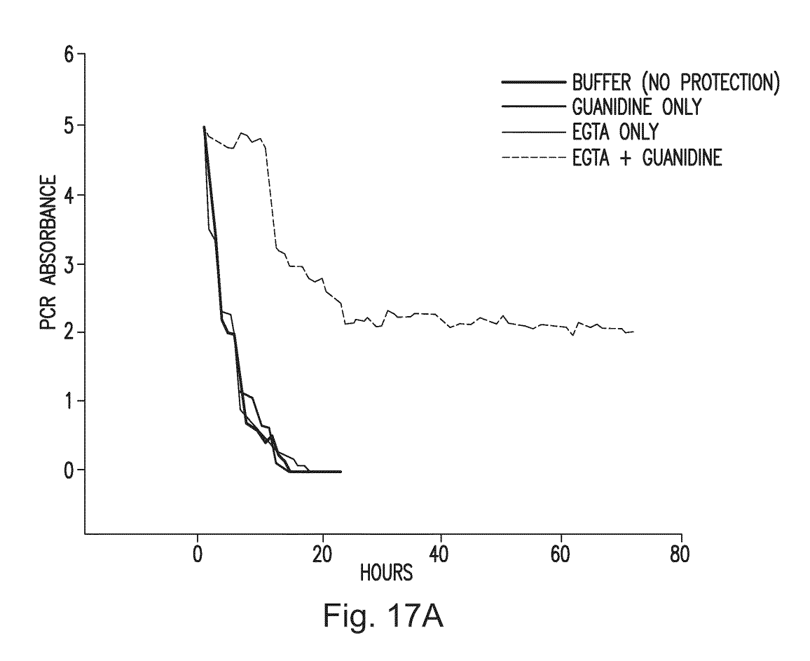

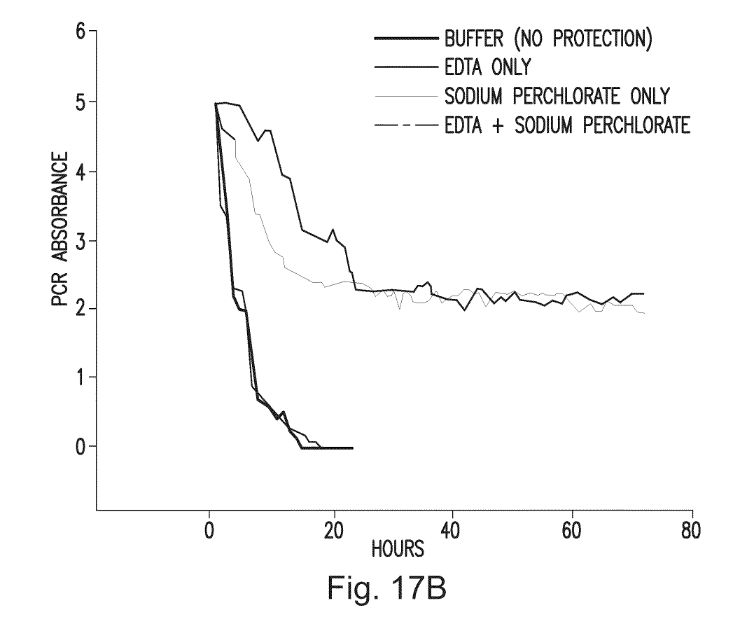

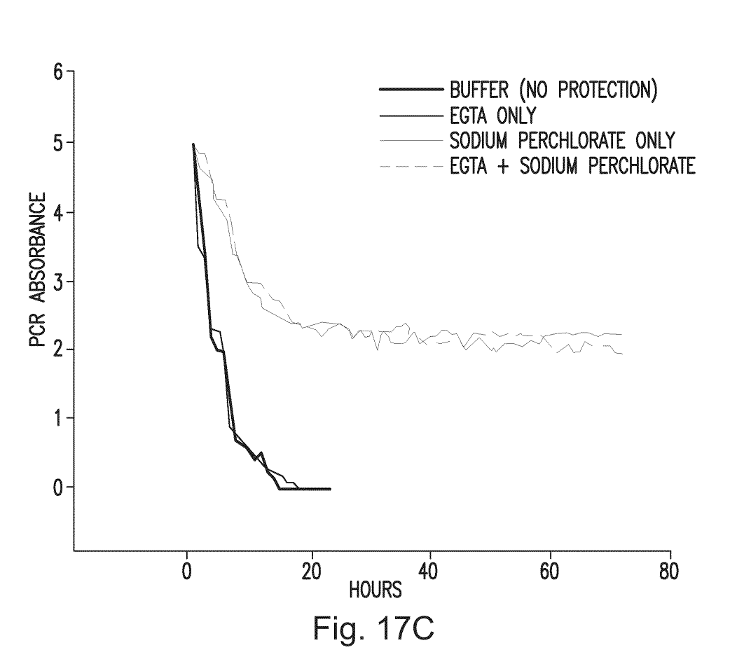

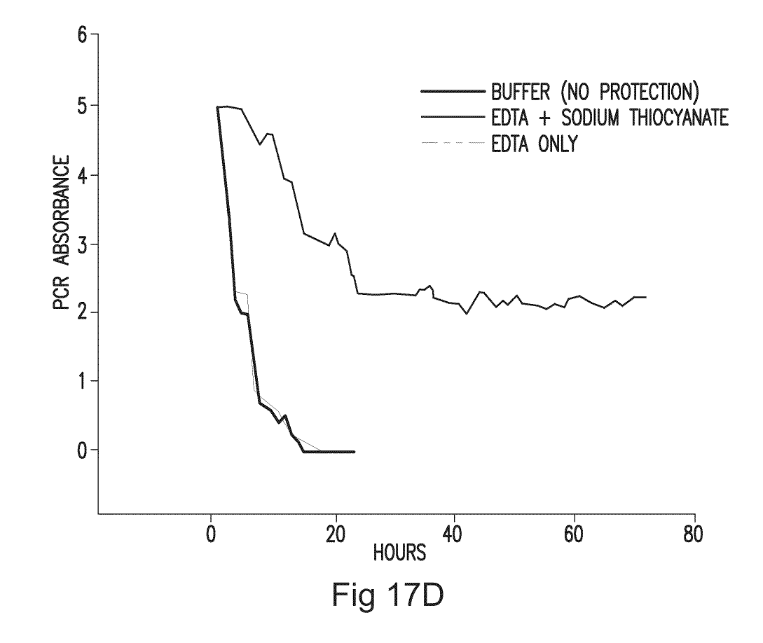

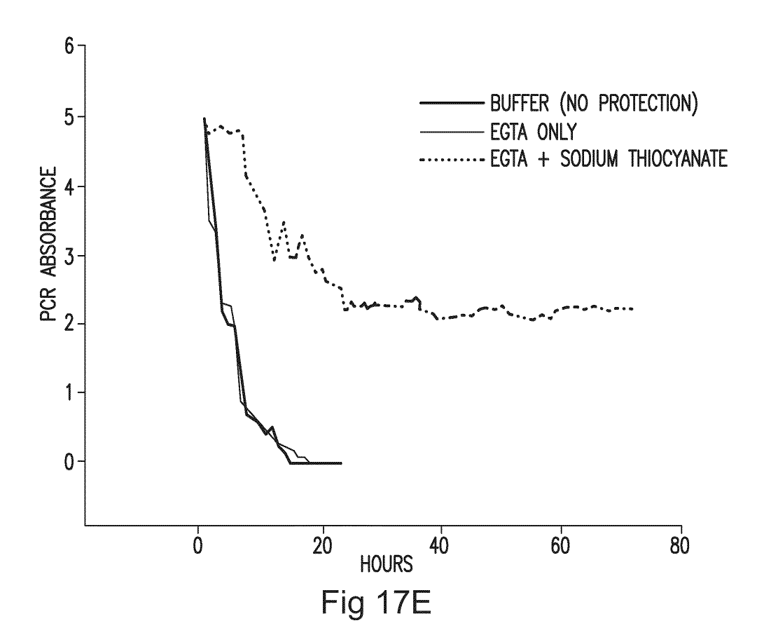

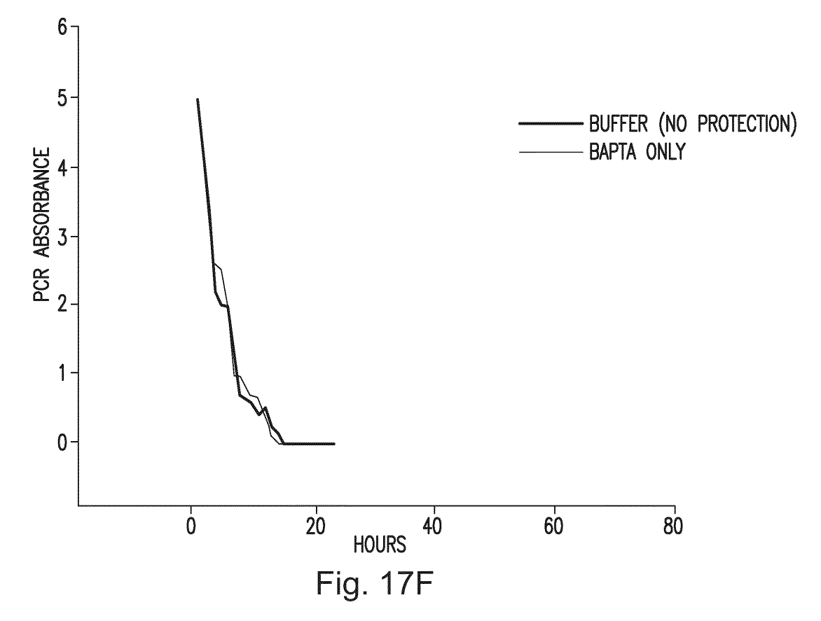

FIG. 17 illustrates the surprising and synergistic effect obtained by the combination of divalent metal chelators and chelator enhancing components in the inventive reagent (i.e., 1 M sodium perchlorate/0.01 M EGTA) in protecting hepatitis B sequences in serum stored at room temperature and subsequently subjected to MD03/06 PCR detection. The protocol run was as above (i.e., as illustrated in FIG. 16). As shown in the figures, compared to the addition of EGTA or sodium perchlorate individually, protection of Hep B sequences is dramatically increased when stabilizing solutions of the present disclosure are used.

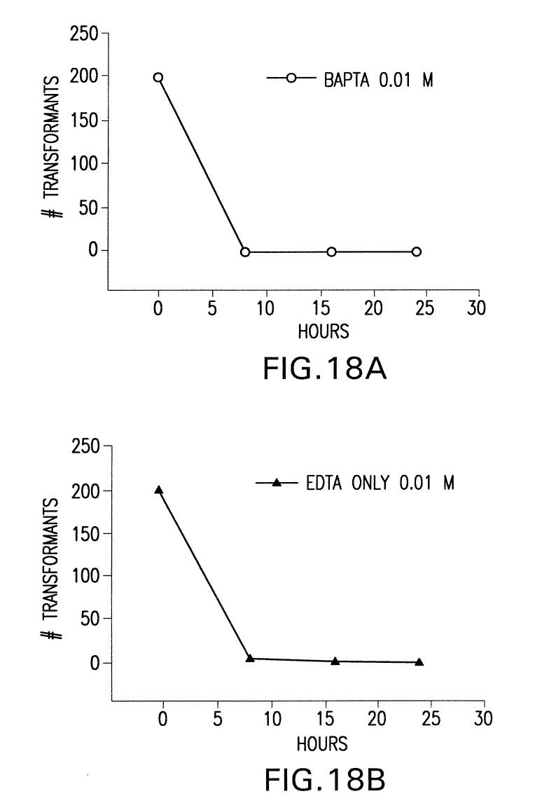

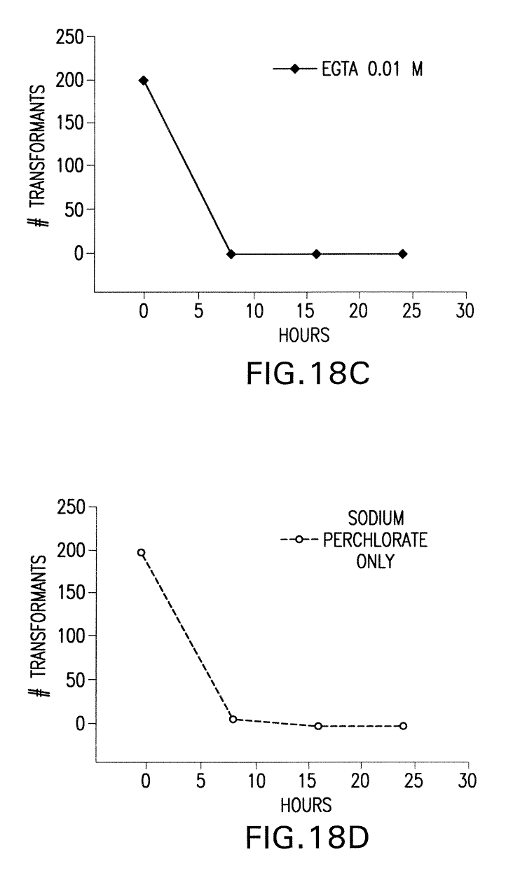

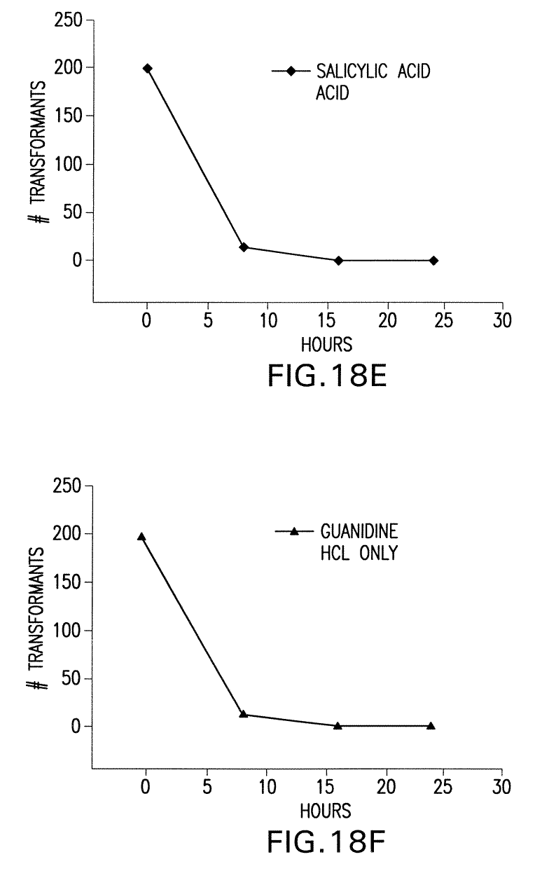

FIG. 18 illustrates the relatively weak stabilizing effect on gonococcal DNA in urine stored at room temperature and subsequently subjected to PCR detection afforded by individual components of reagents of the present disclosure, i.e., divalent metal chelators 0.01 M BAPTA (18A), 0.01 M EDTA (18B), 0.01 M EGTA (18C); and chelator enhancing components 1 M sodium perchlorate (18D), 1 M salicylic acid (18E), 1 M guanidine HCl (18F), 1 M sodium thiocyanate (not shown), and 1 M lithium chloride (not shown). The number of transformants in ten types of urine specimens were tested using a GTT, counted hourly, and then summarized. The standard Gonostat protocol (see Example 2, infra) was employed and illustrated the synergistic effect obtained by the combination of divalent metal chelators and chelator enhancing components in protecting gonococcal DNA in urine stored at room temperature and subsequently subjected to PCR detection.

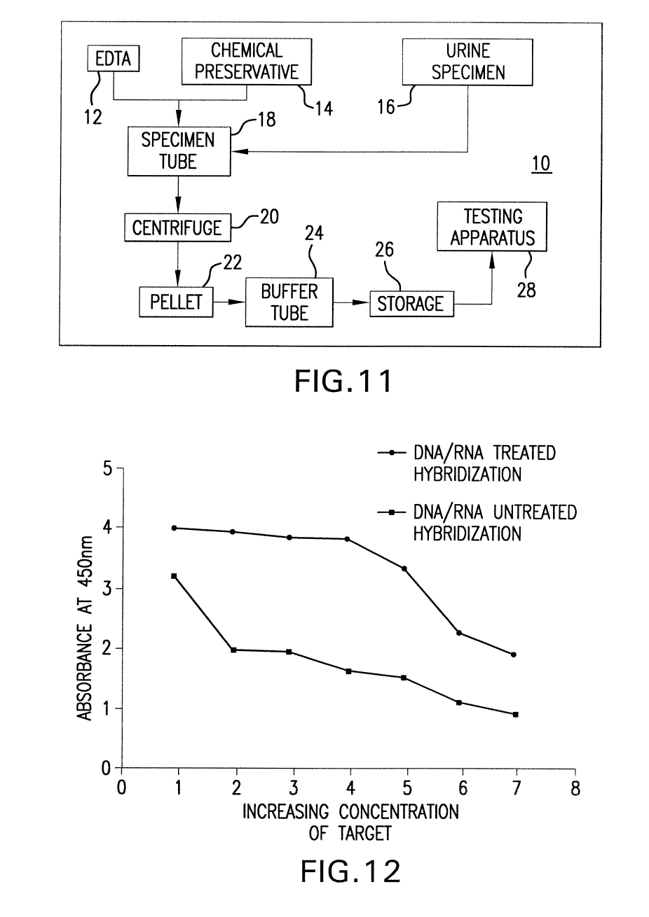

Method 10 shown in FIG. 11 is a specific example embodiment of a method according to the disclosure. This embodiment uses an exemplary protocol to stabilize and test the urine specimens. The protocol is described in Table 1, below. This system produces high yields of DNA/RNA suitable for such testing methods as PCR, restriction fragment length polymorphisms assay (RFLP), and nucleic acid probes from urine specimens.

TABLE 1
1. 10 ml of clean catch urine 16 is added to a specimen test tube 18 containing divalent metal
chelator 12 and chelator enhancing component 14. Test tube is inverted two or three times to
mix the urine.
2. Test tube is transported to laboratory. No refrigeration is necessary. Note: The test tube
should be stored in a cool place and not in direct sunlight.
3. At the laboratory, the test tube is centrifuged 20 at 3200 rpm for 10 minutes.
4. Using a sterile transfer pipette, the pellet 22 at the bottom of the test tube is transferred to
another test tube containing buffer 24. (As little urine as possible should be transferred with
the pellet material.)
5. The buffered material is stored 26 at between 2-8° C. until ready to test 28.
6. The specimen size necessary to run the assay-needs to be validated on the individual test
methodology and individual testing protocol being used.

A small molecule to be stabilized may be a steroid, such as a steroid with pheromone activity. An example of a steroid with pheromone activity is androsterone. A molecule to be stabilized may also be another steroid, such as testosterone or a synthetic (“designer”) steroid such as tetrahydrogestrinone, dehydrochlortestosterone, metandienone, methyltestosterone, androlone, oxandrolone, oxymetholone, or stanozolol, as well as their analogues, precursors, and metabolites. With the increasing concern about the illegal and dangerous use of anabolic steroids among athletes, both amateur and professional, and the consequently increasing use of urine tests to detect such use, there is a need for a reliable method of stabilizing steroids in urine samples for later testing, supplied by methods and compositions according to the present disclosure.

When the molecule to be stabilized is a protein, it may be a protein with any of a variety of biological activities, such as an enzyme, an antibody, a receptor protein, a regulatory protein, a membrane protein, or a structural protein. The protein may be monomeric or multimeric. If the protein is multimeric, methods and compositions according to the present disclosure may be effective in stabilizing its quaternary structure; that is, the specific interaction between the subunits that is required to stabilize the activity of the protein. In some embodiments, a protein may be protected from degradation by way of the ubiquitin system.

A protein to be stabilized may be a protein that is normally degraded by the ubiquitin system (e.g., degradation that catalyzed by activating enzymes E-1, E-2, E-3 in the presence of ATP and the 28S proteasome). A biological fluid in which the nucleic acid, protein, or small molecule is to be stabilized may be, but is not limited to, urine, blood, serum, plasma, amniotic fluid, cerebrospinal fluid, seminal fluid, vaginal fluid, stool, conjunctival fluid, salivary fluid, or sweat. In some embodiments, a biological fluid may be or may comprise urine.

A kit, in some embodiments, may comprise (1) a stabilizing composition according to the present disclosure; (2) a vessel for collecting a biological fluid in which a nucleic acid, protein, or small molecule is to be stabilized; and (3) instructions for use. A vessel may contain a stabilizing composition ready for use; alternatively, a stabilizing composition may be packaged separately from a vessel. In some embodiments, a stabilizing composition for stabilization of a protein and/or a small molecule (e.g., a steroid) may include higher concentrations of divalent metal chelator and chelator enhancing component.

Kits according to the present disclosure, as described above, may be used for testing or screening purposes. When used for testing or screening purposes, such kits may further comprise at least one sample containing the molecule to be stabilized at a known concentration in a stabilizing composition. This sample may be used as a standard or a control in later testing, such as testing of human urine to determine the concentration of testosterone. The kit may include multiple samples containing the molecule to be stabilized at a range of known concentrations, so that a standard curve may be run.

According to some embodiments, a composition may comprise: (1) animal urine; and (2) a stabilizing composition of the present disclosure, such that the animal urine contains a pheromone in sufficient quantity to act as an attractant to an animal of the same species as the animal from which the animal urine comes. Animal urine may be from an animal that is hunted, such as a deer (mule deer, whitetail deer, or other deer), a fox, a bear, a boar, an elk, a moose, or a raccoon in some embodiments. A pheromone may be a steroid, such as androsterone, but compositions of the disclosure are not limited to the stabilization of steroids. Compositions and methods for stabilization of a pheromone may include, in some embodiments, higher concentrations of a divalent metal chelator and/or a chelator enhancing component as desired and/or required, for example, for maximum stabilization of pheromone concentration.

For example, in some embodiments, a method of stabilizing pheromone activity of an animal urine comprising the steps of: (1) providing a fresh animal urine containing pheromone activity; and (2) adding the fresh animal urine to a stabilizing composition of the present disclosure to stabilize the pheromone activity at a level such that the urine containing a stabilizing composition acts as an attractant to an animal of the same species as the animal from which the animal urine comes.

The role of pheromones is described, for example., in B. Rasmussen, “Why Musth Elephants Use Pheromones,” Biologist 50: 195-196 (2003); R. Hudson, “Back to Basics: Expressive Behaviour,” at http://www.deer.rr.ualberta.ca/library/backtobasics/bbcommunication.htm; M. V. Novotny et al., “A Unique Urinary Constituent, 6-Hydroxy-6-Methyl-3-Heptanone, Is a Pheromone That Accelerates Puberty in Female Mice,” Chem. Biol. 6: 377-383 (1999); and “Pheromones: The Chemical Signals for Attraction,” at http://is2.dal.ca/-kcollin2/pheromones.html, all of which are incorporated herein by this reference.

A stabilized fluid may comprise, according to some embodiments, (1) a stabilizing composition for stabilizing a molecule selected from a protein and a small molecule and (2) a bodily fluid from a human or non-human subject, wherein a stabilizing composition may comprise (a) an amount of a divalent metal chelator selected from the group consisting of ethylenediaminetetraacetic acid (EDTA), (ethylenebis(oxyethylenenitrilo))tetraacetic acid (EGTA), and 1,2-bis(2-aminophenoxy)ethane-N,N,N′,N′-tetraacetic acid (BAPTA) and salts thereof in the range of from about 0.001 M to about 2 M, and (b) an amount of at least one chelator enhancing component selected from the group consisting of lithium chloride, guanidinium chloride, guanidinium thiocyanate, sodium salicylate, sodium perchlorate, and sodium thiocyanate in the range of from about 0.1 M to about 10 M. An example bodily fluid is urine, but may be another bodily fluid. As described above, a bodily fluid may be from a human or non-human source.

The disclosure is illustrated by the following Examples. These Examples are included for illustrative purposes only, and are not intended to limit the disclosure.

### Examples

Some specific example embodiments of the disclosure may be illustrated by one or more of the examples provided herein.

### Example 1

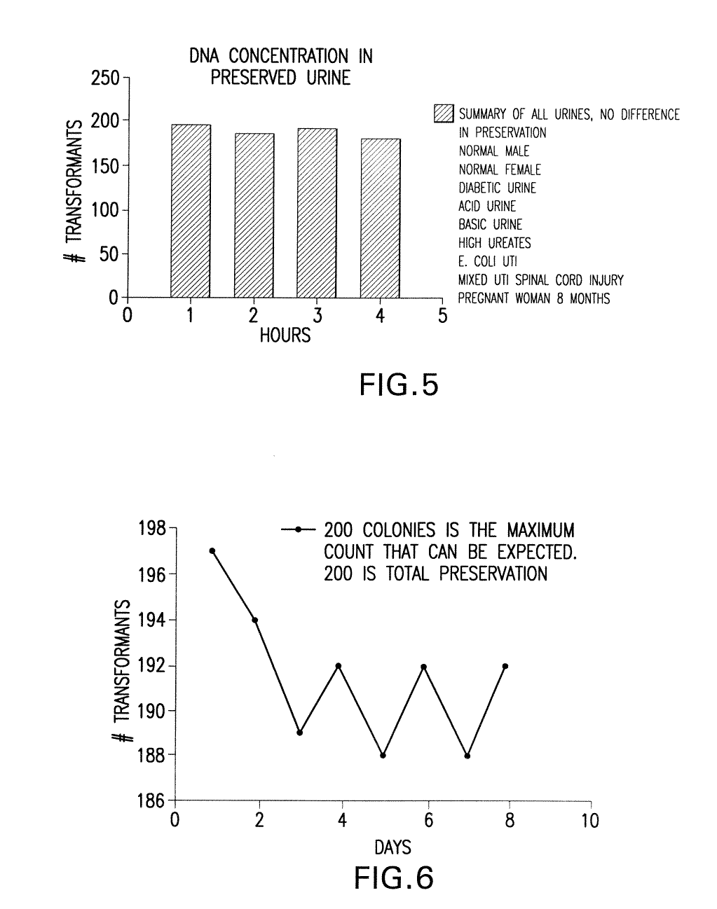

FIG. 5 is a bar graph of DNA concentration in stabilized urine in accordance with the disclosure. The number of transformants in ten types of urine specimens were tested using a GTT, counted hourly, and then summarized. The standard Gonostat protocol (see Example 2, infra) was employed, and the stabilizing composition used was 1 M guanidine HCl/0.01 M EDTA. A count of two hundred colonies demonstrates total stabilization of a specimen. The number of gonococcal transformants in the stabilized urine remained relatively constant approaching two hundred, throughout the four hours of the test. No significant difference in level of stabilization was observed among the different types of urine specimens. Therefore, the disclosure provides, according to some embodiments, nearly total protection for DNA in urine.

### Example 2

FIG. 6 is a graph of eight day GTT serial data on stabilized urine according to the disclosure. 1 pg of gonococcal DNA was spiked into 9 ml of fresh human urine and 1 ml of aqueous stabilizing composition containing 1 M sodium perchlorate and 0.01 M EGTA. 300 μl was spotted onto a lawn of the Gonostat organism at 24 hour intervals for eight days. The plates contained BBL Chocolate II agar and were incubated at 37° C. for 24 hours before readings were taken. The number of colonies observed throughout the eight-day testing period ranged from a low count of one hundred eighty-eight to a high count of one hundred ninety-seven. Thus, methods and compositions of the disclosure, according to some embodiments, stabilize DNA in urine for a longer period of time (e.g., significantly longer) than previously provided.

### Example 3

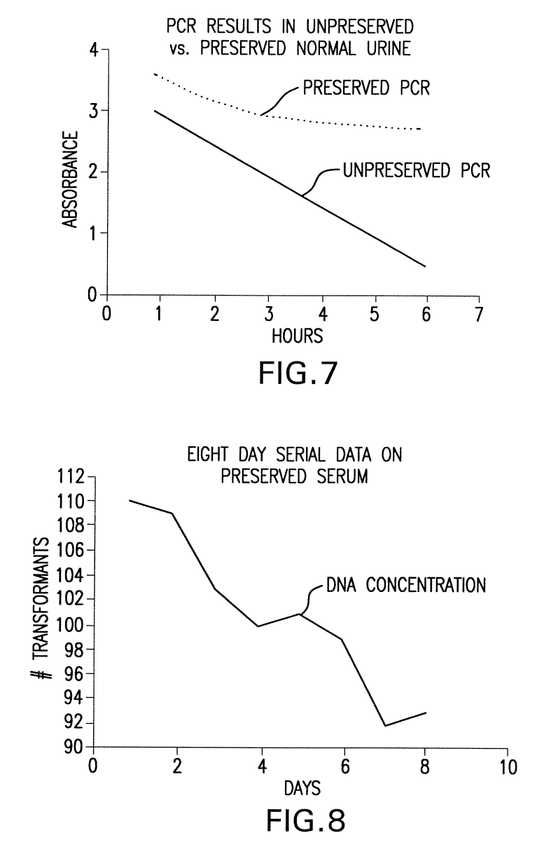

FIG. 7 is a graph comparing PCR results in unstabilized and stabilized normal urine according to the disclosure. A MOMP template to Chlamydia trachomatis was used and amplified using a standard PCR protocol. 200 copies of the MOMP target were spiked into 9 ml of fresh human urine containing 1 M sodium perchlorate and 0.01 M BAPTA. PCR was done each hour for eight hours total. In the unprotected urine, approximately three PCR absorbances were measured one hour after the addition of DNA to the urine. The number of PCR absorbances approached zero by the sixth hour. By contrast, in the stabilized specimen, in excess of three PCR absorbances were measured at the one hour testing. However, approximately three PCR absorbances were still observed by the sixth hour. Therefore, the disclosure stabilizes sufficient DNA and nucleic acid sequences to permit PCR testing well beyond the testing limits of unstabilized urine. The results shown in the Figure are consistent for all types of DNA in a urine specimen.

### Example 4

In some embodiments, methods, systems, and/or reagents of the disclosure may be used for stabilizing other bodily fluids and excretions, such as blood serum. FIG. 8 is a graph of eight day serial data on stabilized serum according to the disclosure. The protocol used was similar to Example 3, except fresh human serum was used. The number of transformant colonies observed throughout the eight-day testing period ranged from a high count of one hundred ten at the one day measurement to a low count of approximately ninety-two at the seven day measurement. In fact, the test results actually showed an increase in transformant colonies between days seven and eight. Thus, methods and compositions of the disclosure, according to some embodiments, stabilize DNA in serum for a longer period of time (e.g., significantly longer) than previously provided.

### Example 5

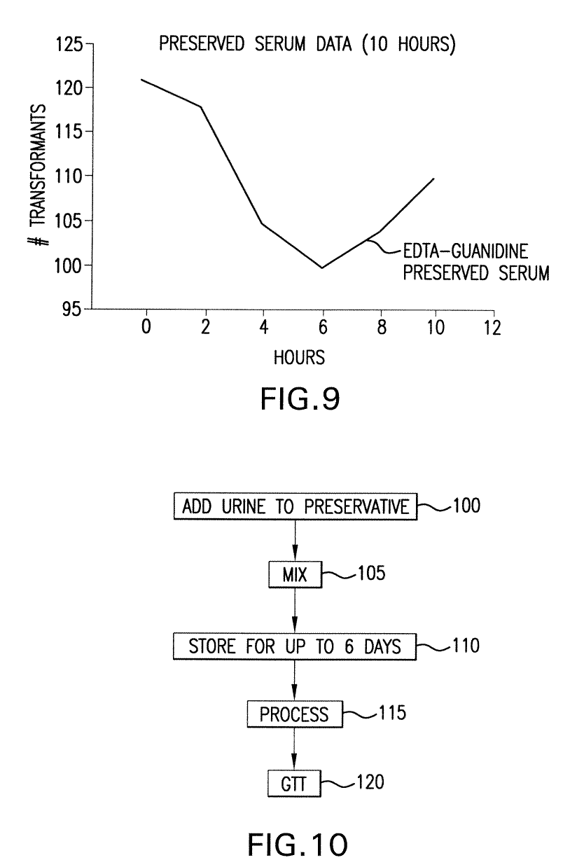

FIG. 9 is a graph of DNA concentration in stabilized serum according to the disclosure. The serum was stabilized with stabilizing composition solution comprising 1 M guanidine HCl/0.01 M EDTA. The protocol used was similar to Example 3, except fresh human serum was used, and the duration time of the study was ten hours. In excess of 120 transformants were measured at the time gonococcal DNA was added to the serum. Approximately 100 transformants were counted at the six hour measurement. However, by the tenth hour, testing indicated that the concentration of biologically active DNA in the stabilized serum had increased to approximately 110 transformant colonies.

### Example 6

### Stabilization of DNA in Simulated Clinical Specimens

In the following experiment, simulated clinical urine specimens were produced and tested for the presence of gonococcal DNA. The chemicals listed in Table 2, below, were added, at the concentrations previously described, to urine specimens from healthy adults, as was EDTA.

A suspension of gonococci was immediately added to each urine specimen. The added gonococci were an ordinary strain of N. gonorrheae, 49191, which was grown overnight on GC agar medium at 37° C. in a 5% CO₂ atmosphere. The N. gonorrheae colonies were picked and suspended in GC buffer. A 1/10 volume of a suspension containing approximately 10 Colony forming units (cfu) per ml was added to the urine. As a positive control, the suspension of gonococci was also added to HEPES buffer.

All simulated clinical specimens and the HEPES controls were tested at time zero, i.e., when the chemicals and gonococci were added. The specimens and controls were also tested after storage at room temperature for six days. This six day period was selected to approximate the maximum time expected between collecting, mailing, and testing patient specimens.

With the exception of urine samples containing SDS and sarkosyl, the simulated specimens and HEPES controls were processed as follows:

1. A 10 ml quantity was centrifuged at 4000 rpm for 30 minutes.

2. The supernatant was decanted, and the pellet was suspended in 1 ml phosphate buffer.

3. The suspension was heated for 10 minutes in a water bath at 60° C.

4. After cooling, the suspension was used in the GTT.

The simulated urine specimens containing SDS-EDTA or sarkosyl-EDTA were processed as follows:

1. Approximately a 2½ volume (approximately 25 ml) of 95% ethyl alcohol was added to the tube with the urine and stabilizing composition. The contents were mixed by inverting the tube several times.

2. The mixture was centrifuged at 4000 rpm for 30 minutes.

3. The pellet was suspended in 10 ml of 70% alcohol and centrifuged.

4. The pellet was then suspended in 1 ml phosphate buffer.

5. The suspension was heated for 10 minutes in a water bath at 60° C.

6. After cooling, the suspension was used in the GTT.

The inoculated urine was stored at room temperature for 6 days prior to testing. The formulations that stabilized (+) or did not stabilize (−) gonococcal DNA in the inoculated urine for six days to approximately the same degree as in the HEPES buffer control are indicated. Although the results of the Gonostat™ assay may be semi-quantitated, the tests were not designed to rank the relative efficacy of the chemical stabilizers. Thus, the results given in Table 2 indicate whether or not the particular chemical stabilized DNA in urine over a six day period to same degree as in the HEPES buffer.

TABLE 2
Stabilizing Composition

Compositions Having Stabilizing Effect
0.01 M EDTA + 1 M Guanidinium Hydrochloride
0.01 M EDTA + 1 M Guanidinium Thiocyanate
0.01 M EDTA + 1 M Lithium Chloride
0.01 M EDTA + 1 M Manganese Chloride
0.01 M EDTA + 1% Sarkosyl
0.01 M EDTA + 1% Sodium Dodecyl Sulfate
0.01 M EDTA + 1 M Sodium Perchlorate
0.01 M EDTA + 1 M Sodium Salicylate
0.01 M EDTA + 1 M Sodium Thiocyanate
Compositions Having No Stabilizing Effect
1 M Sodium Periodate
1 M Trichloroacetic Acid
1 M Urea

The 92% sensitivity exhibited with male urine specimens is comparable to the culture results reported in the literature. In addition, the 88% sensitivity exhibited with female urine specimens exceeds the previously-reported levels.

While some embodiments of the disclosure are directed to the stabilization of gonococcal DNA, it will be readily apparent to one skilled in the art that the disclosure is adaptable for use in stabilizing other types of DNA, such as that of Haemophilus influenzae and Bacillus subtilis. Some embodiments of the disclosure may also be used to stabilize RNA contained in bodily fluid samples. Such stabilized RNA may be used for RNA transcriptase and reverse transcriptase assays for viral segments and human gene sequence testing. Additionally, embodiments of the disclosure may be used to stabilize proteins contained in bodily fluid samples, such as for immunological assays using suitable antibodies.

According to some embodiments, a stabilizing composition may be added to a bodily fluid, e.g., a urine specimen. A bodily fluid, in some embodiments, may be added to a stabilizing composition. According to some embodiments, efficacy may not be adversely impacted by the order of addition. In some embodiments, desirable stabilization (e.g., optimal stabilization) of the DNA may be typically and conveniently achieved by adding a single reagent of the disclosure to the specimen.

### Example 7

### PCR Detection of Penicillinase-producing Neisseria Gonorrheae

The PCR signal-enhancing effect of stabilizing composition reagents of the disclosure is demonstrated by the following example. Four varieties of TEM-encoding plasmids may be found in PPNG. These are the 6.7 kb (4.4 Mda) Asian type, the 5.1 kb (3.2 Mda) African type, the 4.9 kb (3.05-Mda) Toronto type and the 4.8 kb (2.9-Mda) Rio Type. This PCR assay for PPNG takes advantage of the fact that the TEM-1 gene is located close to the end of the transposon Tn2; by the use of one primer in the TEM-1 gene and the other in a sequence beyond the end of Tn2, and common to all four plasmids, a PCR product only from plasmids and not from TEM-1 encoding plasmids was obtained. (Table 3, below) The conditions associated with this protocol were modified to include the DNA/RNA protect reagent in the hybridization and the treated probe was mixed with the 761-bp amplification product per standard PCR protocol. The results were read as absorbance at 450 nanometers.

Materials and Reagents
BBL chocolate 11 agar plates
Sterile Tris Buffer 10 mM Tris (pH 7.4), 1 mM EDTA
0.5-ml Gene Amp reaction tubes
Sterile disposable pasteur pipette tips
Aerosol-resistant tips
PCR master mix: 50 mM KCl, 2 mM MgCl₂, 50 μM each of deoxyribonucleoside triphosphate; 2.5 U of taq Polymerase (Perkin Elmer); 5% glycerol; 50 pmol each of primers PPNG-L and PNG-R (per 100 μl reaction)
Denaturation solution: 1 M Na 5×Denhardt's solution
Prehybridization Solution: 5×SSC (1×SSC is 0.015 M NaCl plus 0.015 M sodium citrate);
5×Denhardt's solution;
0.05% SDS;
0.1% sodium pyrophosphate, and
100 μg of sonicated salmon sperm DNA per ml.
Hybridization Solution
Same as prehybridization solution but without Denhardt's solution and including 200 μl of DNA/RNA protect reagent 1.
1 ml DNA/RNA stabilizing composition (1 M guanidine HCl/0.01 M EDTA)
Avidin-HRP peroxidase complex (Zymed)
Magnetic microparticles (Seradyne)

TABLE 3

| Function | Name | Nucleotide Sequence 5′ to 3′ |
|---|---|---|
| Primer | PPNG-L | AGT TAT CTA CAC GAC GG |
| | | (SEQ ID NO: 1) |
| Primer | PPNG-B | GGC GTA CTA TTC ACT CT |
| | | (SEQ ID NO: 2) |
| Probe | PPNG-C | GCG TCA GAG CCC TAT CTA TAA ACT C |
| | | (SEQ ID NO: 3) |

Methods

Sample preparation: 2 colonies were picked from a chocolate agar plate. Colonies were suspended in Dl water just prior to setting up PCR. The master mix was prepared according to the recipe above. 5 μl of the freshly prepared bacterial suspension was added to 95 μl of master mix. The DNA was liberated and denatured in a thermocycler using three cycles of 3 min at 94° C. and 3 min at 55° C. The DNA was amplified in the thermal cycler by using a two step profile: a 25 s denaturation at 95° C. and a 25 s annealing at 55° C. for a total of thirty cycles. The time was set between the two temperature plateaus to enable the fastest possible annealing between the two temperatures. 15 pmol of labeled (avidin-HRP complex) detection probe PPNG-C was added to the hybridization solution bound to magnetic micro particles with and without stabilizing composition reagent at 37° C. for 1 hour. The control and treated probes were then added to the amplification product and the reaction was colorimetrically detected by absorbance at 450 nm. The signal obtained from the hybridization probes treated with a reagent of the disclosure was found to be significantly higher than the untreated probes.

### Example 8

### Stabilization of Androsterone in Human Urine

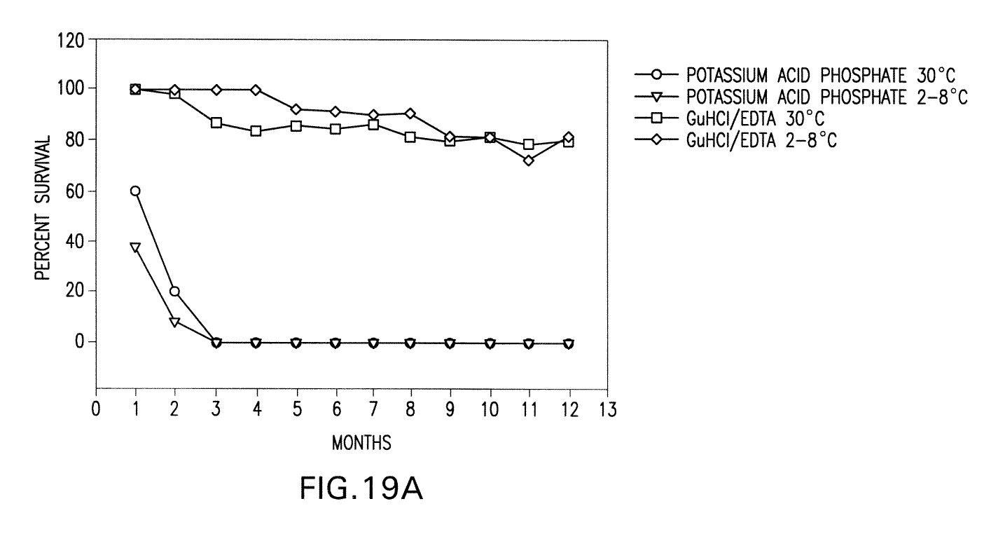

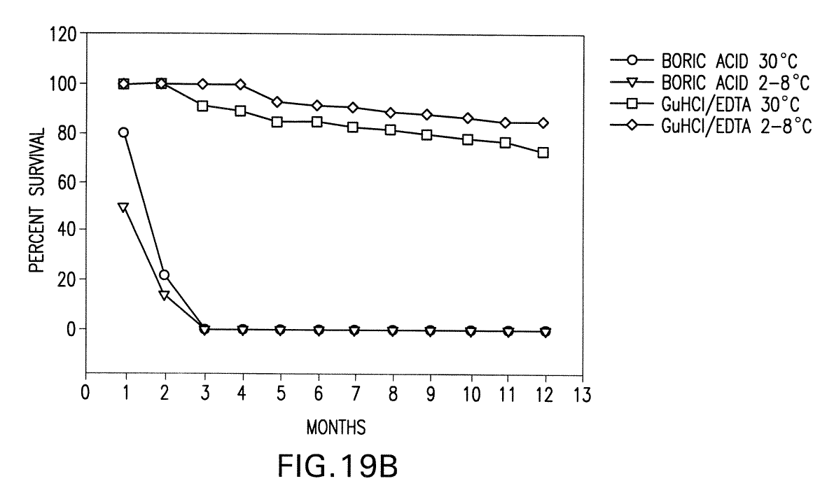

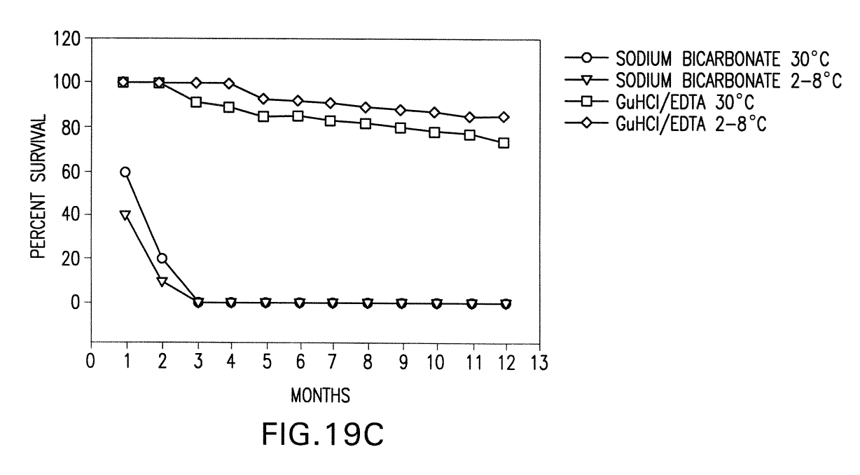

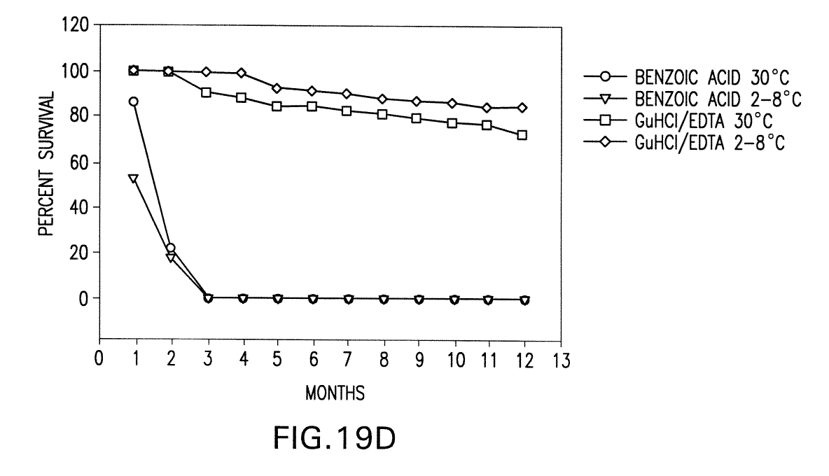

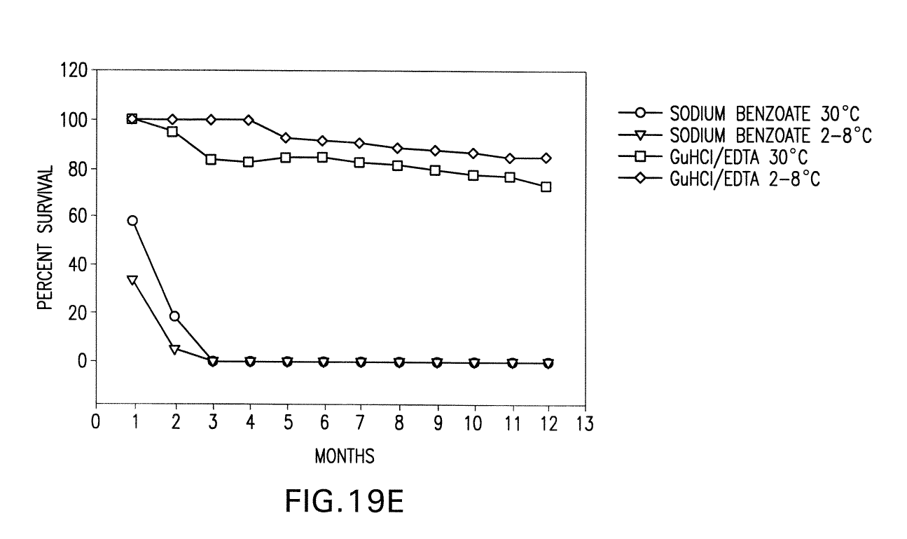

The formulation described above (1 M guanidinium HCl/0.01 M EDTA) was tested to determine its effectiveness in stabilizing the swine pheromone androsterone, a steroid, added to human urine. Human urine was used as a base, with the swine pheromone androsterone added to the solution. Solutions were prepared using the following stabilizers: (1) 1 M guanidinium HCl/0.01 M EDTA; (2) potassium acid phosphate; (3) boric acid; (4) sodium bicarbonate; (5) benzoic acid; and (6) sodium benzoate. One portion of each of the six stabilizing solutions with the androsterone-spiked urine was kept at 8° C. and one portion was kept at 30° C. Solutions were maintained and tested monthly over a 12-month period. Testing was done by turbidity testing of antibody concentration using a spectrophotometer. The results are shown in the five comparison graphs as follows: FIG. 19A: guanidinium

HCl/EDTA (“Gu/HCl/EDTA”) versus potassium acid phosphate; FIG. 19B: guanidinium HCl/EDTA versus boric acid; FIG. 19C: guanidinium HCl/EDTA versus sodium bicarbonate; FIG. 19D: guanidinium HCl/EDTA versus benzoic acid; and FIG. 19E: guanidinium HCl/EDTA versus sodium benzoate.

To summarize, the guanidinium HCl/EDTA solution stabilized the androsterone molecules at or near the 100% level through four months, and over the next eight months maintained the androsterone levels at above 80% of the original concentration. Of the other stabilizing compositions, only one maintained androsterone concentration levels as high at 80% after even one month; none of the others maintained as much as a 20% concentration after two months, and all of the concentrations, other than the guanidinium HCl/EDTA test solution, were reduced to 0% by the third months.

Thus, the guanidinium HCl/EDTA solution stabilized the steroid androsterone in urine over an extended period of time.

### Example 10

### Stabilization of Proteins in Urine

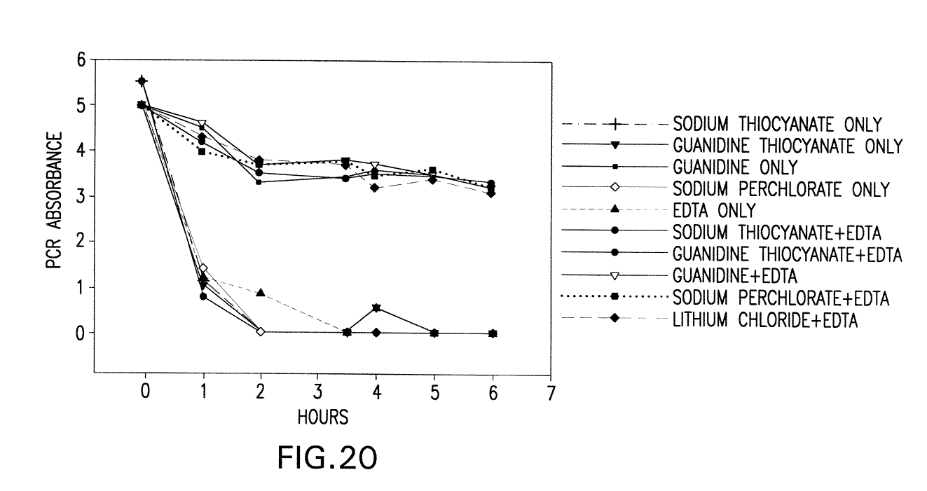

FIG. 20 shows the prevention of degradation of protein AF176555 (calpain) in urine by the ubiquitin-285 proteasome pathway using single agents and combination agents; with chaotropic agents used at 2 M and chelators at 0.1 M. The single agents were sodium thiocyanate, guanidinium thiocyanate, guanidinium HCl, sodium perchlorate, and EDTA. The combination agents were sodium thiocyanate+EDTA, guanidinium thiocyanate+EDTA, guanidinium HCl+EDTA, sodium perchlorate+EDTA, and lithium chloride+EDTA. The results shown in FIG. 20 show that the combination agents were substantially effective in preventing the degradation of calpain over 6 hours in urine; the single agents were substantially ineffective, with degradation occurring by 2 hours in most instances.

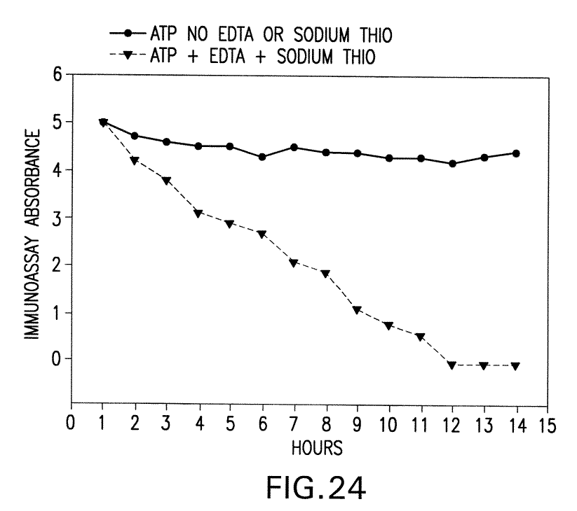

For the results in FIGS. 20-23, the proteins were quantitated by attaching appropriate PCR primers to segments of the protein so that PCR amplification would only occur on undegraded proteins, then performing PCR and quantitating the amount of amplification by absorbance. For the results in FIG. 24, the ATP was quantitated by immunoassay.

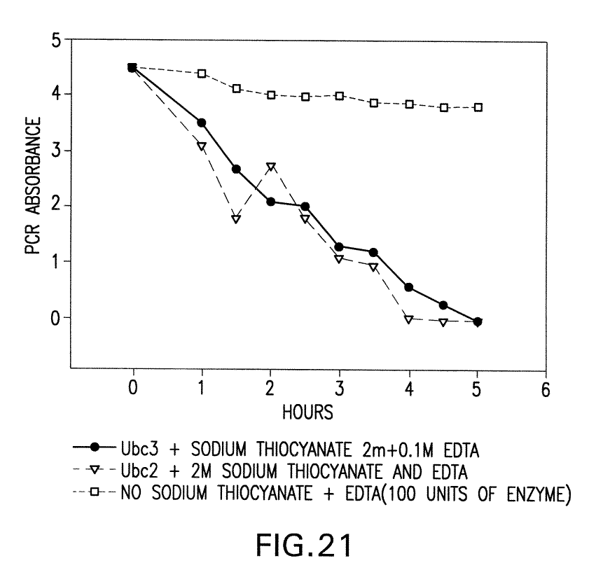

FIG. 21 shows the survival of ubiquitin activating enzymes Ubc2 (E-2) and Ubc3 (E-2) in urine with and without 2M sodium thiocyanate and 0.1 M EDTA. The ubiquitin-activating enzymes survived for a longer period of time without the sodium thiocyanate-EDTA. These results are consistent with protection of proteins that would normally be degraded by the ubiquitin system from degradation by the combination of sodium thiocyanate and EDTA.

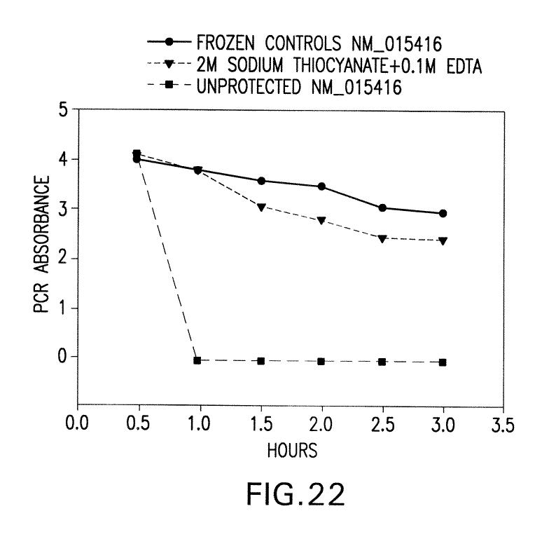

FIG. 22 similarly shows the survival of protein AF068706 (G2AD) from degradation by the ubiquitin system in urine spiked with ubiquitin, activating enzymes E-1, E-2, E-3, ATP, and 28S proteasome by 2 M sodium thiocyanate+0.1 M EDTA compared with frozen controls and unprotected protein. The unprotected protein was degraded rapidly, while the protein protected with 2 M sodium thiocyanate and EDTA was protected nearly as well as frozen controls.

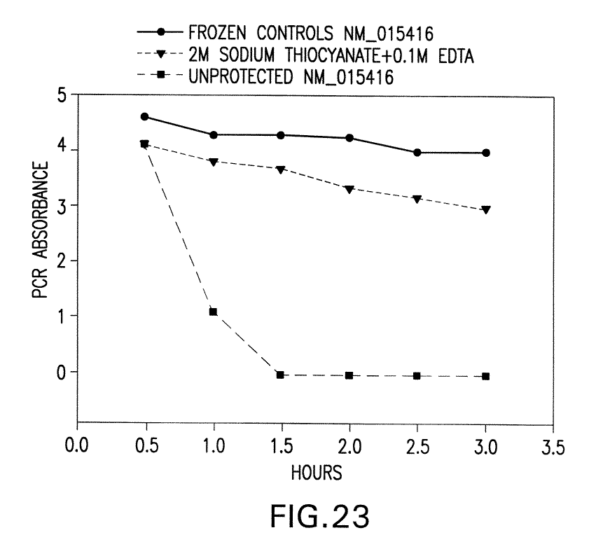

FIG. 23 similarly shows the survival of Protein NM_{—}015416 (cervical cancer proto-oncogene protein p40) from degradation by the ubiquitin system in urine spiked with ubiquitin, activating enzymes E-1, E-2, E-3, ATP, and 28S proteasome by 2 M sodium thiocyanate+0.1 M EDTA compared with frozen controls and unprotected protein. The unprotected protein was degraded rapidly, while the protein protected with 2 M sodium thiocyanate and EDTA was protected nearly as well as frozen controls.

FIG. 24 shows the survival of ATP in urine with and without exposure to 2 M sodium thiocyanate+0.1 M EDTA. ATP is degraded more rapidly in the presence of the 2 M thiocyanate and 0.1 M EDTA. Because ATP is involved in the degradation of proteins via the ubiquitin pathway, this result is consistent with the protection of proteins from degradation by the ubiquitin pathway by these reagents.

The present disclosure provides compositions and methods that provide efficient stabilization of nucleic acids, including DNA and RNA, proteins, including proteins subject to degradation by the ubiquitin system, and small molecules, including steroids, in bodily fluids. The proteins and small molecules are available for participation in specific reactions, including antigen-antibody reactions, enzymatic reactions, and receptor-binding reactions. These compositions and methods are useful in many applications, including diagnostic and forensic applications. They are also useful for providing a source of animal pheromones for hunters and fishermen.

In some embodiments, the volume and/or weight ratio of stabilizing composition to sample (e.g., bodily fluid) may be from about 1:10 to about 10:1, from about 1:10 to about 1:1, and/or from about 1:10 to about 1:5. A stabilizing composition may be combined with a sample at a ratio of from about 10 μg to about 10 mg of stabilizing composition per milliliter and/or gram of sample. A stabilizing composition may be added to a sample to be stabilized (e.g., a vessel containing the sample) according to some embodiments. A sample to be stabilized may be added, in some embodiments, to a stabilizing composition (e.g., a vessel containing stabilizing composition). According to some embodiments, a stabilizing composition and a sample to be stabilized may be added to each other at the same time. For example, both may be added to an otherwise empty mixing vessel.

As will be understood by those skilled in the art, other equivalent or alternative compositions, systems, and methods for stabilizing a cell and/or a macromolecule and/or biomolecule according to embodiments of the present disclosure can be envisioned without departing from the essential characteristics thereof. For example, a stabilizing composition may be prepared and/or used as a solid, a liquid, or a gas (e.g., a vapor). A stabilizing composition, according to some embodiments, may be formulated as a powder, granule, tablet, capsule, gel, liquid, syrup, and/or paste. A stabilizing composition, in some embodiments, may include one or more solvents (e.g., aqueous and/or organic), bases (e.g., purine and/or pyrimidine bases), buffers, salts, surfactants, oxidizing agents, reducing agents, and/or other reagents. A stabilizing composition may be deposited in a sample container by any available method. For example, a stabilizing composition may be coated (e.g., sprayed or spray-dried) onto an inner surface of a sample container before a macromolecule-containing sample is introduced. A stabilizing composition may also be simply placed in a sample container in a solid or liquid form. Alternatively, a stabilizing composition may be kept in a separate container or compartment and only contacted with a sample after the sample has been placed in a sample container. Also, where ranges have been provided, the disclosed endpoints may be treated as exact and/or estimates as desired or demanded by the particular embodiment In addition, it may be desirable in some embodiments to mix and match range endpoints. In some embodiments, the term “about” when applied to a numeric value may refer to that numeric value plus or minus about 1% of that value, plus or minus about 5% of that value, plus or minus about 10% of that value, plus or minus about 25% of that value, and/or plus or minus about 50% of that value. When the numeric value is provided as an endpoint to a range, the term “about” may have more or less flexibility depending on the extent of the range, according to some embodiments. For example, if the range covers a single order of magnitude (e.g., from about 1 to about 10), “about” may have less flexibility (e.g., expanding endpoints by ±5%). For a range that covers several orders of magnitude (e.g., from about 0.1 to about 100), however, the endpoints may have more flexibility (e.g., expanding endpoints by ±50%). In some embodiments, a concentration range that includes the term “up to” (e.g., up to 1 mM of NaCl) may include a lower endpoint that reaches any amount of the material above zero (e.g., any trace of NaCl). The term “up to,” in some embodiments, may contemplate and/or require that some non-zero amount of the specified material is present. These equivalents and alternatives along with obvious changes and modifications are intended to be included within the scope of the present disclosure. The present disclosure is intended to be illustrative, but not limiting, of the scope of the disclosure. The appended claims are similarly intended to be illustrative, but not limiting, of the scope of the disclosure.

Some specific embodiments of the disclosure may be understood, by referring, at least in part, to the following specific example embodiments. These examples illustrate some, but not necessarily all, aspects of some embodiments of the disclosure and additional variations will be apparent to one skilled in the art having the benefit of the present disclosure.

What is claimed is:

1. A method of stabilizing a molecule comprising a biomolecule selected from the group consisting of a nitrite, a carbohydrate, a ketone, a globin, a bilirubin, a lipid, and combinations thereof, the method comprising:
(a) providing a stabilizing solution comprising:
(i) an amount of a divalent metal chelator selected from the group consisting of ethylenediaminetetraacetic acid (EDTA), (ethylenebis(oxyethylenenitrilo))tetraacetic acid (EGTA), and 1,2-bis(2-aminophenoxy)ethane-N,N,N′,N′-tetraacetic acid (BAPTA) and salts thereof in the range of from about 0.001 M to about 2 M; and
(ii) an amount of at least one chelator enhancing component selected from the group consisting of lithium chloride, guanidinium chloride, guanidinium thiocyanate, sodium salicylate, sodium perchlorate, and sodium thiocyanate in the range of from about 0.1 M to about 10M; and
(b) adding the stabilizing solution to the bodily fluid, thus stabilizing the molecule.

2. A method according to claim 1 wherein the biomolecule comprises a globin.

3. A method according to claim 2 wherein the globin is selected hemoglobin, myoglobin, and combinations thereof.

4. A method according to claim 2 wherein the carbohydrate is selected from the group consisting of glucose, fructose, pyruvate, lactate, glycogen, and combinations thereof.

5. A method according to claim 1 wherein the biomolecule comprises a nitrate.

6. A method according to claim 1 wherein the biomolecule comprises a ketone.

7. A method according to claim 6 wherein the ketone IS selected from the group consisting of acetoacetate, acetone, and combinations thereof.

8. A method according to claim 1 wherein the biomolecule comprises a bilirubin.

9. A method according to claim 8 wherein the bilirubin is selected from the group consisting of bilirubin, urobilinogen, urobilin, and combinations thereof.

10. A method according to claim 1 wherein the bodily fluid is selected from the group consisting of urine, blood, serum, plasma, amniotic fluid, cerebrospinal fluid, seminal fluid, vaginal fluid, stool, conjunctival fluid, salivary fluid, and sweat.

11. A method according to claim 1 wherein the bodily fluid is urine.

12. A method according to claim 1 wherein the stabilizing composition further includes at least one enzyme inactivating component selected from the group consisting of manganese chloride, sarkosyl, and sodium dodecyl sulfate in the range of up to about 5% molar concentration.

13. A method according to claim 1 wherein the concentration of divalent metal chelator is at least 0.01 M and the concentration of chelator enhancing component is at least 1.0 M in the stabilizing solution.

14. A stabilizing composition for stabilizing a molecule selected from the group consisting of a nitrite, a carbohydrate, a ketone, a globin, a bilirubin, a lipid, and combinations thereof, the stabilizing composition comprising:
(a) an amount of a divalent metal chelator selected from the group consisting of ethylenediaminetetraacetic acid (EDTA), (ethylenebis(oxyethylenenitrilo))tetraacetic acid (EGTA), and 1,2-bis(2-aminophenoxy)ethane-N,N,N′,N′-tetraacetic acid (BAPTA) and salts thereof in the range of from about 0.001 M to about 2 M; and
(b) an amount of at least one chelator enhancing component selected from the group consisting of lithium chloride, guanidinium chloride, guanidinium thiocyanate, sodium salicylate, sodium perchlorate, and sodium thiocyanate in the range of from about 0.1 M to about 10 M.

15. A stabilizing composition according to claim 14 further comprising at least one enzyme inactivating component selected from the group consisting of manganese chloride, sarkosyl, and sodium dodecyl sulfate in the range of up to about 5% molar concentration.

16. A stabilizing composition according to claim 14 wherein the biomolecule comprises a globin.

17. A stabilizing composition according to claim 16 wherein the globin is selected hemoglobin, myoglobin, and combinations thereof.

18. A stabilizing composition according to claim 16 wherein the carbohydrate is selected from the group consisting of glucose, fructose, pyruvate, lactate, glycogen, and combinations thereof.

19. A stabilizing composition according to claim 14 wherein the biomolecule comprises a nitrate and/or a ketone.

20. A stabilizing composition according to claim 19 wherein the ketone is selected from the group consisting of acetoacetate, acetone, and combinations thereof.

21. A stabilizing composition according to claim 14 wherein the biomolecule comprises a bilirubin selected from the group consisting of bilirubin, urobilinogen, urobilin, and combinations thereof.

22. A stabilizing composition according to claim 14 further comprising at least one enzyme inactivating component selected from the group consisting of manganese chloride, sarkosyl, and sodium dodecyl sulfate in the range of up to about 5% molar concentration.

23. A stabilizing composition according to claim 14 wherein the concentration of divalent metal chelator is at least 0.01 M and the concentration of chelator enhancing component is at least 1.0 M.

24. A stabilized fluid comprising:
(a) a stabilizing composition for stabilizing a molecule selected from the group consisting of a a nitrite, a carbohydrate, a ketone, a globin, a bilirubin, a lipid, and combinations thereof, the stabilizing composition comprising:
(i) an amount of a divalent metal chelator selected from the group consisting of ethylenediaminetetraacetic acid (EDTA), (ethylenebis(oxyethylenenitrilo))tetraacetic acid (EGTA), and 1,2-bis(2-aminophenoxy)ethane-N,N,N′,N′-tetraacetic acid (BAPTA) and salts thereof in the range of from about 0.001 M to about 2 M; and
(ii) an amount of at least one chelator enhancing component selected from the group consisting of lithium chloride, guanidinium chloride, guanidinium thiocyanate, sodium salicylate, sodium perchlorate, and sodium thiocyanate in the range of from about 0.1 M to about 10M; and
(b) a bodily fluid from a human or non-human subject.


SEQUENCE LISTING (3 sequences)

SEQ ID NO: 1 - DNA, 17 bp, Artificial Sequence
  Synthetic
agttatctacacgacgg

SEQ ID NO: 2 - DNA, 17 bp, Artificial Sequence
  Synthetic
ggcgtactattcactct

SEQ ID NO: 3 - DNA, 25 bp, Artificial Sequence
  Synthetic
gcgtcagacccctatctataaactc